(19) United States
(12) Patent Application Publication
(10) Pub. No.: US 2023/0174545 A1
(43) Pub. Date: Jun. 8, 2023

(54) HETEROCYCLIC COMPOUNDS AS BET INHIBITORS

(71) Applicant: Nuvation Bio Inc., New York, NY (US)

(72) Inventors: Son Minh PHAM, San Francisco, CA (US); Jayakanth KANKANALA, Plymouth, MN (US); Chris P. MILLER, San Mateo, CA (US); Jeremy D. PETTIGREW, Vancouver (CA)

(21) Appl. No.: 17/921,939

(22) PCT Filed: Apr. 28, 2021
(86) PCT No.: PCT/US2021/029738

§ 371 Date: Oct. 27, 2022

Related U.S. Application Data
(60) Provisional application No. 63/017,543, filed on Apr. 29, 2020.

(51) Int. Cl. C07D 491/048; C07F 5/04; A61P 35/00

(52) U.S. Cl. CPC C07D 491/048; C07F 5/04; A61P 35/00

(57) ABSTRACT

Novel bromodomain and extraterminal domain (BET) inhibitors and to therapeutic methods of treating conditions and diseases using these novel BET inhibitors are provided.

### CROSS-REFERENCE TO RELATED APPLICATION

This application claims priority to U.S. provisional application No. 63/017,543, filed Apr. 29, 2020, entitled “HETEROCYCLIC COMPOUNDS AS BET INHIBITORS,” the contents of which are incorporated herein by reference in their entirety for all purposes.

### FIELD

The present disclosure relates to novel bromodomain and extraterminal domain (BET) inhibitors and methods of treating conditions and diseases using these novel BET inhibitors.

### BACKGROUND

Epigenetic dysregulation has a crucial role in driving aberrant gene expressions leading to various types of cancers. Many components involved in epigenetic regulation have been attractive targets for therapeutic interventions. Among them, the bromodomain and extra-terminal (BET) family of proteins attracted much attention in recent years. The BET family proteins include BRD2, BRD3, BRD4, and the testis-specific BRDT. Via their bromodomains (BRDs), they bind with a high affinity to acetylation motifs, including acetylated histones in chromatin, thereby regulating gene transcription. The genes regulated by BET family proteins include many important oncogenes responsible for cell survival and cell cycle progression.

BET proteins are emerging targets in cancer, directly regulating the expression of oncogenes in hematological and solid tumors. BRD4, in addition to occupying gene promoters, has a strong preference for enhancers and super-enhancers in key driver genes such as c-MYC (Loven et al, Cell 2013; 153(2):320-34). BET family proteins have also been implicated in mediating acute inflammatory responses through the canonical NF-KB pathway (Huang et al., Mol. Cell. Biol. 29: 1375-1387 (2009)) resulting in the upregulation of genes associated with the production of cytokines (Nicodeme et al., Nature 468: 1119-1123, (2010)). In addition, bromodomain function has been implicated in kidney disease (Zhang, et al., J. Biol. Chem. 287: 28840-28851 (2012)). BRD2 function has also been linked to a predisposition for dyslipidemia or improper regulation of adipogenesis, elevated inflammatory profiles and increased susceptibility to autoimmune diseases (Denis, Discovery Medicine 10: 489-499 (2010)). The human immunodeficiency virus utilizes BRD4 to initiate transcription of viral RNA from stably integrated viral DNA (Jang et al., Mol. Cell, 19: 523-534 (2005)). BET bromodomain inhibitors have also been shown to reactivate HIV transcription in models of latent T cell infection and latent monocyte infection (Banerjee, et al., J. Leukocyte Biol. doi:10.1189/jlb.0312165). BRDT has an important role in spermatogenesis (Matzuk, et al., Cell 150: 673-684 (2012)).

Due to this potential as an epigenetic target, a number of small molecule compounds that inhibit the function of BET family proteins have been developed, and many of them have demonstrated promising anti-cancer activities with both solid and hematologic malignancies in preclinical studies. This has led to several early-phase clinical trials. Included among these are R06870810 (formerly TEN-010), ZEN003694, BMS-986158, CPI-0610, I-BET762, OTX015, FT-1101, INCB054329, PLX51107, GS-5829, and ABBV-075. While these efforts are promising, there is need for better selectivity and improved durability of BET inhibitors that provide enhanced efficacy while reducing toxicity related to off-target effects. The present disclosure relates to novel BET inhibitors.

### BRIEF SUMMARY

In one aspect, provided is a compound of Formula (I):

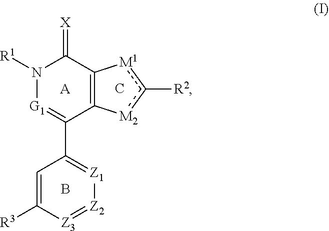

or a tautomer or isomer thereof, or a pharmaceutically acceptable salt of any of the foregoing, wherein [symbol] , X, G₁, R¹, R², R³, M¹, M², Z₁, Z₂, and Z₃ are as detailed herein.

In some embodiments, the compounds provided herein are BET inhibitors that selectively target and covalently bind the protein of interest. In some embodiments, the BET inhibitors comprise a compound of the Formula (I), or any related formula, such as (II), (IIa-1) to (IIa-14), (III), or (IIIa-1) to (IIIa-14), or a tautomer or isomer thereof, or a pharmaceutically acceptable salt of any of the foregoing.

In some embodiments, provided herein is a pharmaceutical composition comprising a compound of Formula (I), or any related formula, such as (II), (IIa-1) to (IIa-14), (III), or (IIIa-1) to (IIIa-14), or a tautomer or isomer thereof, or a pharmaceutically acceptable salt of any of the foregoing, in combination with at least one pharmaceutically acceptable carrier, diluent, or excipient.

In some embodiments, use of a compound having the structure of Formula (I), or any related formula, such as (II), (IIa-1) to (IIa-14), (III), or (IIIa-1) to (IIIa-14), or a tautomer or isomer thereof, or a pharmaceutically acceptable salt of any of the foregoing, for the manufacture of a medicament is provided.

In some embodiments, provided herein is a method of treating a disease mediated by the BET family of proteins in an individual. In some embodiments, such method comprises administering to the subject an effective amount of a compound of Formula (I), or any related formula, such (II), (IIa-1) to (IIa-14), (III), or (IIIa-1) to (IIIa-14), or a tautomer or isomer thereof, or a pharmaceutically acceptable salt of any of the foregoing, or a pharmaceutical composition comprising the same, at a frequency and for duration sufficient to provide a beneficial effect to the subject.

In some embodiments, provided herein is a method of treating or preventing disorders that are ameliorated by inhibition of BET. In some embodiments, such method comprises of administering to the subject a therapeutically effective amount of a compound of Formula (I), or any related formula, such as (II), (IIa-1) to (IIa-14), (III), or (IIIa-1) to (IIIa-14), or a tautomer or isomer thereof, or a pharmaceutically acceptable salt of any of the foregoing, alone, or in combination with a pharmaceutically acceptable carrier.

In another aspect, the methods are directed to methods of treating or preventing an inflammatory disease or cancer or AIDS. In some embodiments, such methods comprise of administering to the subject a therapeutically effective amount of a compound of Formula (I), or any related formula, such as (II), (IIa-1) to (IIa-14), (III), or (IIIa-1) to (IIIa-14), or a tautomer or isomer thereof, or a pharmaceutically acceptable salt of any of the foregoing, alone, or in combination with a pharmaceutically acceptable carrier.

In another aspect, provided herein is the use of a compound of Formula (I), or any related formula, such as (II), (IIa-1) to (IIa-14), (III), or (IIIa-1) to (IIIa-14), or a tautomer or isomer thereof, or a pharmaceutically acceptable salt of any of the foregoing, alone or in combination with a second active pharmaceutical agent, in the manufacture of a medicament for treating or preventing conditions and disorders disclosed herein, with or without a pharmaceutically acceptable carrier.

In another aspect, a method of synthesis is provided for a compound having the structure of Formula (I), or any related formula, such as (II), (IIa-1) to (IIa-14), (III), or (IIIa-1) to (IIIa-14), or a tautomer or isomer thereof, or a pharmaceutically acceptable salt of any of the foregoing, as detailed herein.

### DETAILED DESCRIPTION

### Definitions

“Alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkylene” as used herein refers to the same residues as alkyl but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Alkynyl” as used herein refers to an unsaturated linear or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

“Carbonyl” refers to the group C═O.

“Cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted by more than one halogen, it may be referred to by using a prefix corresponding to the number of halogen moieties attached, e.g., dihaloaryl, dihaloalkyl, trihaloaryl etc. refer to aryl and alkyl substituted by two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, pyridazinyl, thiophenyl, furanyl, thiazolyl, pyrrolyl, pyrazolyl, oxazolyl, isooxazolyl, imidazolyl, quinolyl, isoquinolyl, benzimidazolyl, benzpyrazolyl, benzotriazolyl, indole, benzothiazyl, benzoxazolyl, benzisoxazolyl, imidazopyridinyl and the like.

“Heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, dihydrooxazolyl, dihydroisoxazolyl, dioxolanyl, morpholinyl, dioxanyl, tetrahydrothiophenyl, and the like.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same or different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

Term “BET” refers to bromodomain and extraterminal domain family.

As used herein “BRD” refers to one or more bromodomain extraterminal domain family proteins (BRD2, BRD3, BRD4, and BRDT).

“Disease” specifically includes any unhealthy condition of an animal or part thereof. “Optional” or “optionally” means that the subsequently described event or circumstance may or may not occur, and that the description includes instances where the event or circumstance occurs and instances in which it does not.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary-butylacetic acid, lauryl sulfuric acid, gluconic acid, glutamic acid, hydroxynaphthoic acid, salicylic acid, stearic acid, muconic acid, and the like.

In addition, pharmaceutically acceptable salts may be formed when an acidic proton present is capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Therapeutically effective amount” means that amount which, when administered to an animal for treating a disease, is sufficient to affect such treatment for the disease.

“Treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the disclosure contemplate any one or more of these aspects of treatment.

Compounds that have identical molecular formulae but differ in the nature or sequence of bonding of their atoms or in the arrangement of their atoms in space are termed “isomers.” Isomers that differ in the nature or sequence of bonding of their atoms are termed “constitutional isomers.” Isomers that differ only in the arrangement of their atoms in space are termed “stereoisomers.” Stereoisomers that are not mirror images of one another are termed “diasteromers” and stereoisomers that are mirror images are termed “enantiomers” or sometimes “optical isomers.” Stereoisomers that are superimposable upon their mirror images are termed “achiral” and those not superimposable are termed “chiral.” A carbon atom bonded to four different groups is termed a “chiral center” or alternatively an “asymmetric carbon.”

When a compound has a chiral center, a pair of enantiomers of opposite chirality is possible. An enantiomer can be characterized by the absolute configuration of its chiral center and described by the R- and S-sequencing rules of Cahn and Prelog (i.e., as (R)- and (S)-isomers) or by the manner in which the molecule rotates the plane of polarized light and designated as dextrorotatory or levorotatory (i.e., as (+)- and (−)-isomers, respectively). A chiral compound can exist as either individual enantiomer or as a mixture thereof. A mixture containing equal proportions of the enantiomers is termed a “racemic mixture” or “racemate” and may be described as the (RS)- or (±)-mixture thereof. Unless indicated otherwise, the description or naming of a particular compound in the specification and claims is intended to include both individual enantiomers and mixtures, racemic or otherwise, thereof. Conventions for stereochemical nomenclature, methods for the determination of stereochemistry and the separation of stereoisomers are well-known in the art (see discussion in Chapter 4 of “Advanced Organic Chemistry”, 3rd edition March, Jerry, John Wiley and Sons, New York, 1985).

### Compounds

In some embodiments, provided is a compound of Formula (I):

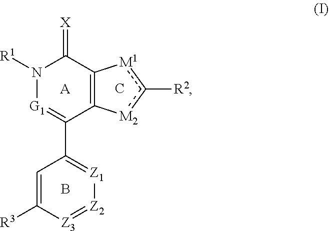

or a tautomer or isomer thereof, or a pharmaceutically acceptable salt of any of the foregoing, wherein:
each [symbol] is independently a single bond or double bond;

### X is O or S;

R¹ is hydrogen, C₁-C₃ alkyl, —(C₁-C₃ alkylene)OH, C₁-C₃ haloalkyl, or C₃-C₄ cycloalkyl;
G₁ is CR^{a} or N, wherein:

R^{a} is hydrogen, halogen, C₁-C₆ alkyl, or C₁-C₆ haloalkyl;

Z₁ is C—W₁—R^{c}, wherein:

each W₁ is independently —O— or —NR^{w1}—, wherein:
R^{w1} is hydrogen, C₃-C₆ cycloalkyl, or C₁-C₄ alkyl optionally substituted by oxo, —OH, or halogen, and

R^{c} is independently C₃_C₆ cycloalkyl, 4- to 6-membered heterocyclyl, C₆-C₁₄ aryl, or 5- or 6-membered heteroaryl, each of which is independently optionally substituted by R^{c1}, wherein each R^{c1} is independently halogen, C₁-C₄ alkyl, C₃-C₆ cycloalkyl, 3- to 6-membered heterocyclyl, cyano, oxo, C₁-C₄ alkoxy, C₁-C₄ haloalkoxy, C₁-C₄ haloalkyl, —OR¹⁰, —NR¹⁰R¹¹, —C(O)NR¹⁰R¹¹, —NR¹⁰C(O)R¹¹, —S(O)₂R¹⁰, —NR¹⁰S(O)₂R¹¹, or —S(O)₂NR¹⁰R¹¹;

Z₂ is C—W₂—R^{d} or N, wherein:

W₂ is —O—, —NR^{w2}—, or a bond, wherein:
R^{w2} is hydrogen, C₃-C₆ cycloalkyl, or C₁-C₄ alkyl optionally substituted by oxo, —OH, or halogen, and

R^{d} is independently hydrogen, halogen, cyano, 3- to 6-membered heterocyclyl, or C₁-C₄ alkyl;

Z₃ is C—R^{e} or N, wherein:

R^{e} is independently hydrogen, halogen, cyano, 3- to 6-membered heterocyclyl, or C₁-C₄ alkyl;

M¹ is O or CR^{1a};
M² is O or CR^{2a}, provided that

(1) when M¹ is O, then the [symbol] adjacent to M¹ is a single bond and the [symbol] adjacent to M² is a double bond,

(2) when M² is O, then the [symbol] adjacent to M² is a single bond and the [symbol] adjacent to M¹ is a double bond, and

(3) at least one of M¹ and M² is O;

R^{1a} and R^{2a} are each independently hydrogen, halogen, C₁-C₄ alkyl, C₃-C₆ cycloalkyl, 3- to 6-membered heterocyclyl, 5- to 10-membered heteroaryl, cyano, C₁-C₄ haloalkyl, C₁-C₄ alkoxy, C₁-C₄ haloalkoxy, —OR¹⁰, —NR¹⁰R¹¹, —C(O)OR¹⁰, —C(O)NR¹⁰R¹¹, —NR^{10C}(O)R¹¹, —S(O)₂R¹⁰, —NR¹⁰S(O)₂R¹¹ or —S(O)₂NR¹⁰R¹¹, each of which is independently optionally substituted by R¹²;
R² is hydrogen, halogen, C₃-C₆ cycloalkyl, 3- to 6-membered heterocyclyl, 5- to 10-membered heteroaryl, cyano, C₁-C₄ alkoxy, C₁-C₄ haloalkoxy, —OR¹⁰, —NR¹⁰R¹¹, —C(O)OR¹⁰, —C(O)NR¹⁰R¹¹, —NR¹⁰C(O)R¹¹, —S(O)₂R¹⁰, —NR¹⁰S(O)₂R¹¹, or —S(O)₂NR¹⁰R¹¹, each of which is independently optionally substituted by R¹²;
R³ is —(CH₂)ₘNR¹³S(O)₂R¹⁴, wherein m is 0, 1, 2 or 3; C₃-C₆ cycloalkyl optionally substituted by halogen, oxo, —CN, or —OH; or C₁-C₄ alkyl substituted by halogen, oxo, —CN, or —OH, provided that when R³ is —(CH₂)ₘNR¹³S(O)₂R¹⁴, then R² is halogen, C₃-C₆ cycloalkyl, 3- to 6-membered heterocyclyl, 5- to 10-membered heteroaryl, cyano, C₁-C₄ alkoxy, C₁-C₄ haloalkoxy, —OR¹⁰, —NR¹⁰R¹¹, —C(O)OR¹⁰, —C(O)NR¹⁰R¹¹, —NR¹⁰C(O)R¹¹, —S(O)₂R¹⁰, —NR¹⁰S(O)₂R¹¹, or —S(O)₂NR¹⁰R¹¹, each of which is independently optionally substituted by R¹²;
R¹⁰ and R¹¹ are each independently hydrogen, C₁-C₄ alkyl, C₁-C₄ alkoxy, C₁-C₄ alkenyl, C₃-C₆ cycloalkyl, 3- to 6-membered heterocyclyl, —(C₁-C₃ alkylene) C₃-C₆ cycloalkyl, —(C₁-C₃ alkylene) 3- to 6-membered heterocyclyl, —NR¹⁵R¹⁶, or —C(O)R¹², wherein each of R¹⁰ and R¹¹ is independently optionally substituted by halogen, oxo, —CN, —CF₃, —OH, —NR¹³R¹⁴, —C(O)NR¹³R¹⁴, or C₁-C₄ alkyl optionally substituted by halogen, oxo, —CN, —CF₃, or —OH,

or R¹⁰ and R¹¹ are taken together with the atom or atoms to which they are attached to form a 3- to 6-membered heterocyclyl ring optionally substituted by halogen, oxo, —CN, —CF₃, —OH, or C₁-C₄ alkyl optionally substituted by halogen, oxo, —CN, or —OH;

each R¹² is independently halogen, cyano, C₁-C₄ haloalkyl, C₁-C₄ alkoxy, C₁-C₄ haloalkoxy, —OR¹⁵, —NR¹⁵R¹⁶, —C(O)NR¹⁵R¹⁶, —NR ¹⁵C(O)R¹⁶, —S(O)₂R¹⁵, —NR¹⁵S(O)₂R¹⁶, —S(O)₂NR¹⁵R¹⁶, C₃-C₆ cycloalkyl, 3- to 6-membered heterocyclyl, or C₁-C₄ alkyl, each of which is independently optionally substituted by halogen, oxo, —CF₃, —CN, —OH, —NR¹³R¹⁴, or —NR¹³C(O)R¹⁴;
R¹³ and R¹⁴ are independently hydrogen, C₁-C₄ alkyl C₃-C₆ cycloalkyl, or 3- to 6-membered heterocyclyl, each of which is independently optionally substituted by halogen, oxo, —CN, or —OH,

or R¹³ and R¹⁴ are taken together with the atom or atoms to which they are attached to form a 3- to 6-membered heterocyclyl ring optionally substituted by halogen, oxo, —CN, —OH, or C₁-C₄ alkyl optionally substituted by halogen, oxo, —CN, or —OH; and

each R¹⁵ and R¹⁶ are independently hydrogen, C₁-C₄ alkyl, C₃-C₆ cycloalkyl, or 3- to 6-membered heterocyclyl, each of which is independently optionally substituted by halogen, oxo, —CN, or —OH,

or R¹⁵ and R¹⁶ are taken together with the atoms to which they are attached to form a 3- to 6-membered heterocyclyl ring optionally substituted by halogen, oxo, —CN, —OH, or C₁-C₄ alkyl optionally substituted by halogen, oxo, —CN, or —OH.

In some embodiments of compound of Formula (I), or a tautomer or isomer thereof, or a pharmaceutically acceptable salt of any of the foregoing, M¹ is O. In some embodiments, M¹ is CR^{1a}. In some embodiments, R^{1a} is hydrogen, halogen, or C₁-C₄ alkyl optionally substituted by R¹². In some embodiments, R^{1a} is hydrogen. In some embodiments, R^{1a} is halogen such as fluoro or chloro. In some embodiments, R^{1a} is C₁-C₄ alkyl such as methyl or ethyl. In some embodiments, M¹ is CR^{1a} and R^{1a} is hydrogen.

In some embodiments of compound of Formula (I), or a tautomer or isomer thereof, or a pharmaceutically acceptable salt of any of the foregoing, M² is O. In some embodiments, M² is CR^{2a}. In some embodiments, R^{2a} is hydrogen, halogen, or C₁-C₄ alkyl optionally substituted by R¹². In some embodiments, R^{2a} is hydrogen. In some embodiments, R^{2a} is halogen such as fluoro or chloro. In some embodiments, R^{2a} is C₁-C₄ alkyl such as methyl or ethyl. In some embodiments, M² is CR^{2a} and R^{2a} is hydrogen.

In some embodiments, M¹ is O and M² is CR^{2a}. In some embodiments, M¹ is O and M² is CR^{2a}, wherein R^{2a} is hydrogen. In some embodiments, M¹ is CR^{1a} and M² is O. In some embodiments, M¹ is CR^{1a}, wherein R^{1a} is hydrogen, and M² is O.

In some embodiments, provided is a compound of Formula (II),

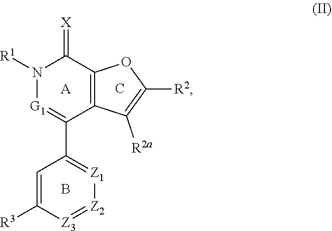

or a tautomer or isomer thereof, or a pharmaceutically acceptable salt of any of the foregoing, wherein X, G₁, R¹, R², R³, R^{2a}, Z₁, Z₂, and Z₃ are as detailed herein for Formula (I).

In some embodiments, provided is a compound of any one of Formula (IIa-1) to (IIa-14):

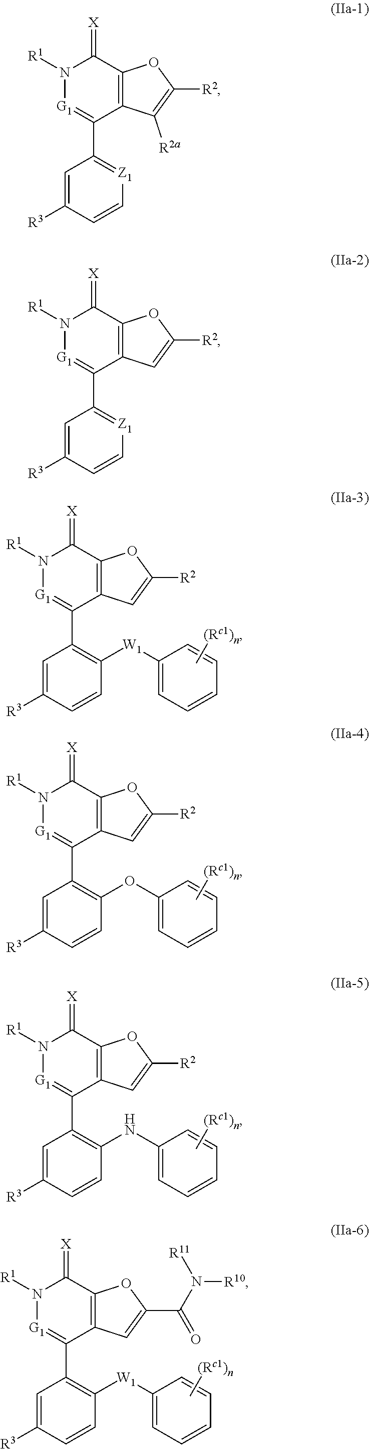

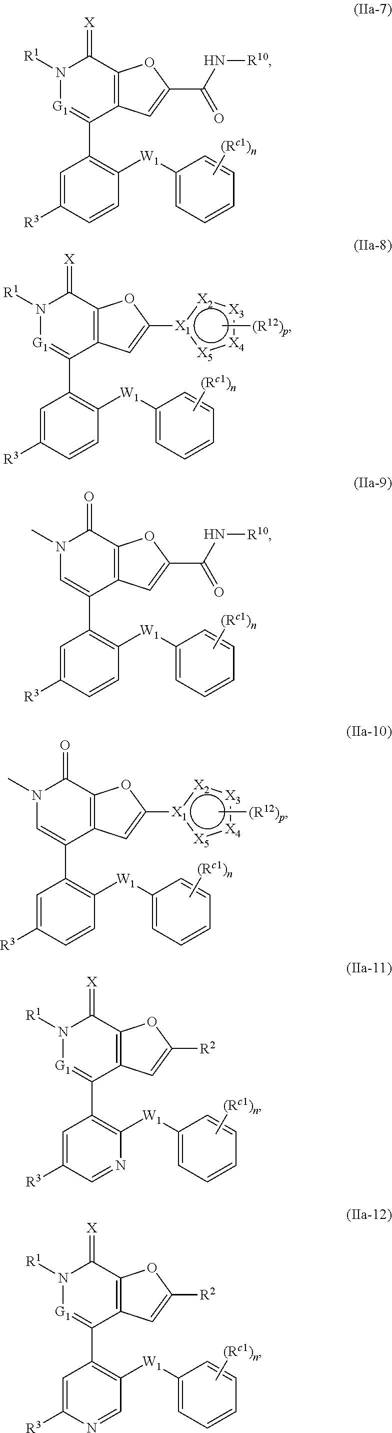

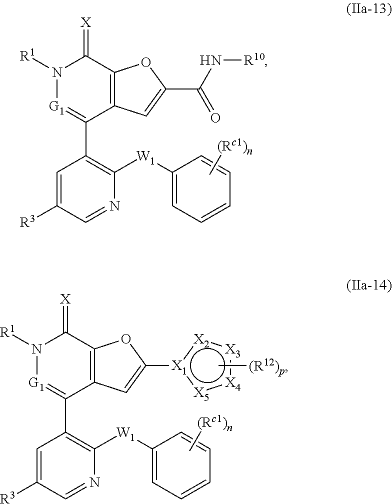

or a tautomer or isomer thereof, or a pharmaceutically acceptable salt of any of the foregoing, wherein X, G₁, R¹, R², R³, R^{2a}, R^{c1}, R¹⁰, R¹¹, R¹², Z₁ and W₁ are as detailed herein for Formula (I);
n is 0, 1, 2, 3, 4, or 5;
p is 0, 1, 2, 3, or 4;

### X₁ is N or C; and

X₂, X₃, X₄ and X₅ are each independently C, CH, CR¹², S, O, N, NR¹², or NH.
In some embodiments, a compound of Formula (I) is of Formula (IIa-1). In some embodiments, a compound of Formula (I) is of Formula (IIa-2). In some embodiments, a compound of Formula (I) is of Formula (IIa-3). In some embodiments, a compound of Formula (I) is of Formula (IIa-4). In some embodiments, a compound of Formula (I) is of Formula (IIa-5). In some embodiments, a compound of Formula (I) is of Formula (IIa-6). In some embodiments, a compound of Formula (I) is of Formula (IIa-7). In some embodiments, a compound of Formula (I) is of Formula (IIa-8). In some embodiments, a compound of Formula (I) is of Formula (IIa-9). In some embodiments, a compound of Formula (I) is of Formula (IIa-10). In some embodiments, a compound of Formula (I) is of Formula (IIa-11). In some embodiments, a compound of Formula (I) is of Formula (IIa-12). In some embodiments, a compound of Formula (I) is of Formula (IIa-13). In some embodiments, a compound of Formula (I) is of Formula (IIa-14).

In some embodiments, provided is a compound of Formula (III),

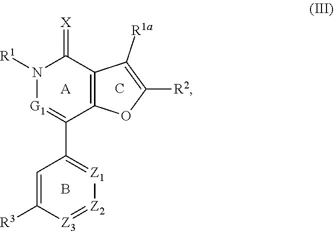

or a tautomer or isomer thereof, or a pharmaceutically acceptable salt of any of the foregoing, wherein X, G₁, R¹, R², R³, R^{1a}, Z¹, Z₂, and Z₃ are as detailed herein for Formula (I).

In some embodiments, provided is a compound of any one of Formula (IIIa-1) to (IIIa-14):

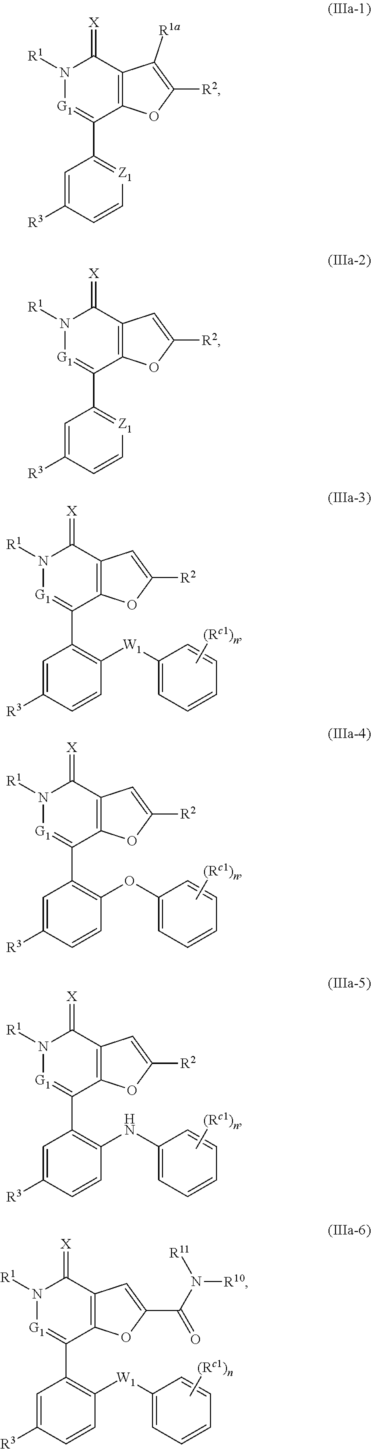

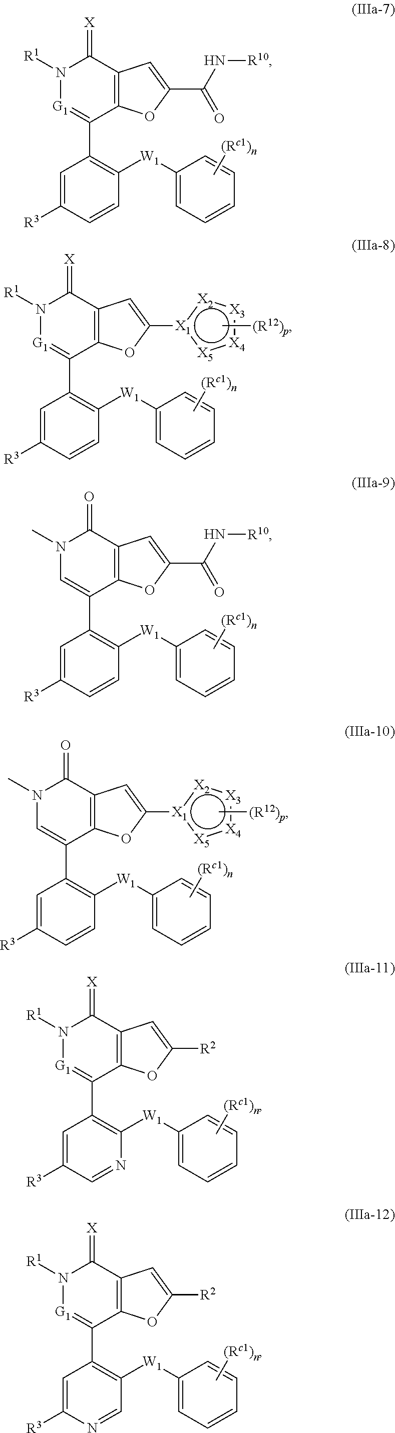

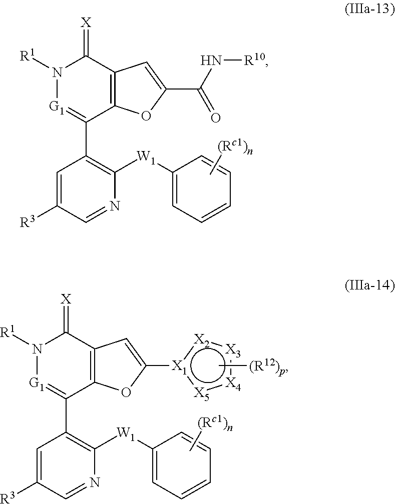

or a tautomer or isomer thereof, or a pharmaceutically acceptable salt of any of the foregoing, wherein X, G₁, R¹, R², R³, R^{2a}, R^{c1}, R¹⁰, R¹¹, R¹², Z₁ and W₁ are as detailed herein for Formula (I);
n is 0, 1, 2, 3, 4, or 5;
p is 0, 1, 2, 3, or 4;

### X₁ is Nor C;

X₂, X₃, X₄ and X₅ are each independently C, CH, CR¹², S, O, N, NR¹², or NH.
In some embodiments, a compound of Formula (I) is of Formula (IIIa-1). In some embodiments, a compound of Formula (I) is of Formula (IIIa-2). In some embodiments, a compound of Formula (I) is of Formula (IIIa-3). In some embodiments, a compound of Formula (I) is of Formula (IIIa-4). In some embodiments, a compound of Formula (I) is of Formula (IIIa-5). In some embodiments, a compound of Formula (I) is of Formula (IIIa-6). In some embodiments, a compound of Formula (I) is of Formula (IIIa-7). In some embodiments, a compound of Formula (I) is of Formula (IIIa-8). In some embodiments, a compound of Formula (I) is of Formula (IIIa-9). In some embodiments, a compound of Formula (I) is of Formula (IIIa-10). In some embodiments, a compound of Formula (I) is of Formula (IIIa-11). In some embodiments, a compound of Formula (I) is of Formula (IIIa-12). In some embodiments, a compound of Formula (I) is of Formula (IIIa-13). In some embodiments, a compound of Formula (I) is of Formula (IIIa-14).

Specific values described herein are values for a compound of Formula (I) or any related formula where applicable, such as Formula (II), (IIa-1) to (IIa-14), (III), or (IIIa-1) to (IIIa-14), or a pharmaceutically acceptable salt or pharmaceutically acceptable tautomer thereof. It is to be understood that two or more values may combined. Thus, it is to be understood that any variable for a compound of Formula (I) or any related formula may be combined with any other variable for a compound of Formula (I) or any related formula the same as if each and every combination of variables were specifically and individually listed.

In some embodiments of a compound of Formula (I) or any related formula, or a tautomer or isomer thereof, or a pharmaceutically acceptable salt of any of the foregoing, X is O.

In some embodiments of a compound of Formula (I) or any related formula, or a tautomer or isomer thereof, or a pharmaceutically acceptable salt of any of the foregoing, R¹ is hydrogen, C₁-C₃ alkyl, —(C₁-C₃ alkylene)OH, C₁-C₃ haloalkyl or C₃-C₄ cycloalkyl. In some embodiments, R¹ is hydrogen. In some embodiments, R¹ is C₁-C₃ alkyl, such as methyl, ethyl, n-propyl, or isopropyl. In some embodiments, R¹ is methyl. In some embodiments, R¹ is C₃-C₄ cycloalkyl, such as cyclopropyl or cyclobutyl. In some embodiments, R¹ is cyclopropyl. In some embodiments, R¹ is —(C₁-C₃ alkylene)OH, such as methanol, ethanol, 1-propanol, or 2-propanol.

In some embodiments of a compound of Formula (I) or any related formula, or a tautomer or isomer thereof, or a pharmaceutically acceptable salt of any of the foregoing, G₁ is N. In some embodiments, G₁ is CR^{a}. In some embodiments, R^{a} is hydrogen. In some embodiments, R^{a} is C₁-C₄ alkyl such as methyl or ethyl. In some embodiments, G₁ is CR^{a} and R^{a} is hydrogen.

In some embodiments of a compound of Formula (I) or any related formula, or a tautomer or isomer thereof, or a pharmaceutically acceptable salt of any of the foregoing, R² is hydrogen. In some embodiments, R² is halogen, C₃-C₆ cycloalkyl, 3- to 6-membered heterocyclyl, 5- to 10-membered heteroaryl, cyano, C₁-C₄ alkoxy, C₁-C₄ haloalkoxy, —OR¹⁰, —NR¹⁰R¹¹, —C(O)OR¹⁰, —C(O)NR¹⁰R¹¹, —NR¹⁰C(O)R¹¹, —S(O)₂R¹⁰, —NR¹⁰S(O)₂R¹¹, or —S(O)₂NR¹⁰R¹¹, each of which is independently optionally substituted by R¹². In some embodiments, R² is hydrogen, —C(O)NR¹⁰R¹¹, or 5- to 10-membered heteroaryl, wherein the —C(O)NR¹⁰R¹¹ and 5- to 10-membered heteroaryl are each optionally substituted by R¹². In some embodiments, R² is —C(O)NR¹⁰R¹¹ or 5- to 10-membered heteroaryl, wherein the —C(O)NR¹⁰R¹¹ and 5- to 10-membered heteroaryl are each optionally substituted by R¹². In some embodiments, R² is —OR¹⁰ such as —OCH₃ or —OCF₃. In some embodiments, R² is —OCF₃. In some embodiments, R² is —OCH₃. In some embodiments, R² is cyano. In some embodiments, R² is halogen such as fluoro or chloro. In some embodiments, R² is —C(O)NR¹⁰R¹¹ which is optionally substituted by R¹². In some embodiments, when R² is hydrogen, —C(O)NR¹⁰R¹¹, or 5- to 10-membered heteroaryl, wherein the —C(O)NR¹⁰R¹¹ and 5-to 10-membered heteroaryl are each optionally substituted by R¹², then R³ is C₃-C₆ cycloalkyl optionally substituted by halogen, oxo, —CN, or —OH or C₁-C₄ alkyl substituted by halogen, oxo, —CN, or —OH. In some embodiments, when R² is hydrogen, —C(O)NR¹⁰R¹¹, or 5- to 10-membered heteroaryl, wherein the —C(O)NR¹⁰R¹¹ and 5- to 10-membered heteroaryl are each optionally substituted by R¹², then R³ is C₁-C₄ alkyl substituted by halogen, oxo, —CN, or —OH. In some embodiments, when R² is hydrogen or —C(O)NR¹⁰R¹¹ which is optionally substituted by R¹², then R³ is C₃-C₆ cycloalkyl optionally substituted by halogen, oxo, —CN, or —OH or C₁-C₄ alkyl substituted by halogen, oxo, —CN, or —OH. In some embodiments, when R² is hydrogen or —C(O)NR¹⁰R¹¹ which is optionally substituted by R¹², then R³ is C₁-C₄ alkyl substituted by halogen, oxo, —CN, or —OH. In some embodiments, when R² is hydrogen, then R³ is C₃-C₆ cycloalkyl optionally substituted by halogen, oxo, —CN, or —OH or C₁-C₄ alkyl substituted by halogen, oxo, —CN, or —OH. In some embodiments, when R² is hydrogen, then R³ is C₁-C₄ alkyl substituted by halogen, oxo, —CN, or —OH. In some embodiments, when R² is —C(O)NR¹⁰R¹¹ which is optionally substituted by R¹², then R³ is C₃-C₆ cycloalkyl optionally substituted by halogen, oxo, —CN, or —OH or C₁-C₄ alkyl substituted by halogen, oxo, —CN, or —OH. In some embodiments, when R² is —C(O)NR¹⁰R¹¹ which is optionally substituted by R¹², then R³ is C₁-C₄ alkyl substituted by halogen, oxo, —CN, or —OH. In some embodiments, R² is —C(O)NR¹⁰R¹¹ which is optionally substituted by R¹², wherein R¹⁰ and R¹¹ are each independently hydrogen, C₁-C₄ alkyl, or C₃-C₆ cycloalkyl, or R¹⁰ and R¹¹ are taken together with the atom or atoms to which they are attached to form a 3- to 6-membered heterocyclyl ring optionally substituted by halogen. In some embodiments, R² is —C(O)NR¹⁰R¹¹ which is optionally substituted by R¹², wherein R¹⁰ and R¹¹ are each independently hydrogen or C₁-C₄ alkyl. In some embodiments, R² is

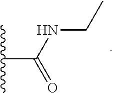 [SMILES: *C(=O)NCC]

In some embodiments, R² is

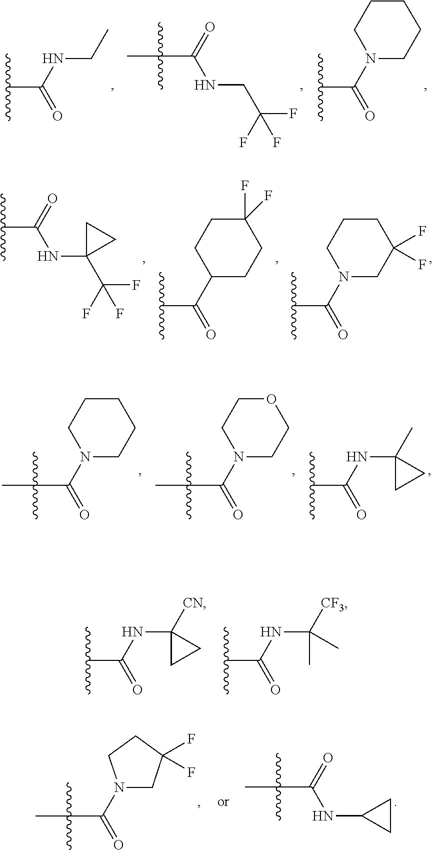

In some embodiments, R² is 5- to 10-membered heteroaryl optionally substituted by R¹². In some embodiments, R² is 5- to 10-membered heteroaryl which is unsubstituted. In some embodiments, R² is a 5- or 6-membered heteroaryl optionally substituted by R¹². In some embodiments, R² is a 5- or 6-membered heteroaryl which is unsubstituted. In some embodiments, R² is a 6-membered heteroaryl optionally substituted by R¹². In some embodiments, R² is a 6-membered heteroaryl which is unsubstituted. In some embodiments, R² is a 5-membered heteroaryl optionally substituted by R¹². In some embodiments, R² is a 5-membered heteroaryl which is unsubstituted. In some embodiments, R² is

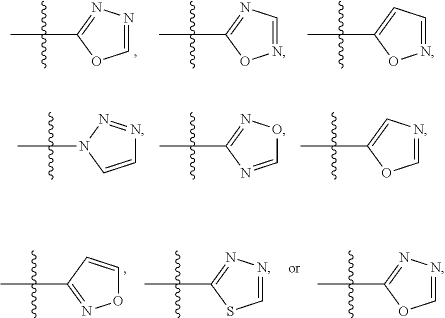

each of which is independently optionally substituted by R¹². In some embodiments, R² is

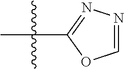 [SMILES: *c1nnco1]

which is optionally substituted by R¹². In some embodiments, R² is

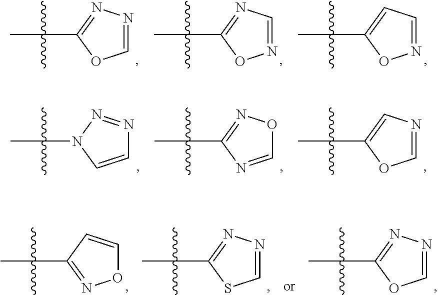

each of which is optionally substituted by R¹², wherein each R¹² is independently C₁-C₄ haloalkyl, C₁-C₄ alkoxy, —NR¹⁵R¹⁶ or C₁-C₄ alkyl. In some embodiments, R² is

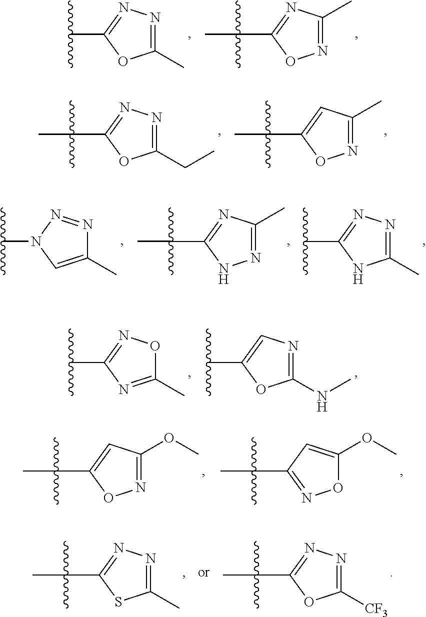

In some embodiments, R² is

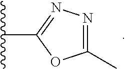 [SMILES: *c1nnc(C)o1]

In some embodiments, R² is

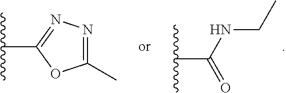

In some embodiments of a compound of Formula (I) or any related formula, or a tautomer or isomer thereof, or a pharmaceutically acceptable salt of any of the foregoing, R³ is —(CH₂)ₘNR¹³S(O)₂R¹⁴ or C₁-C₄ alkyl substituted by halogen, oxo, —CN or —OH. In some embodiments, R³ is C₃-C₆ cycloalkyl optionally substituted by halogen, oxo, —CN, or —OH; or C₁-C₄ alkyl substituted by halogen, oxo, —CN or —OH. In some embodiments, R³ is —(CH₂)ₘNR¹³S(O)₂R¹⁴. In some embodiments, m is 0. In some embodiments, m is 1. In some embodiments, m is 2. In some embodiments, m is 3. In some embodiments, R³ is —(CH₂)ₘNR¹³S(O)₂R¹⁴, wherein m is 0. In some embodiments, R³ is —(CH₂)ₘNR¹³S(O)₂R¹⁴, wherein m is 0 and R¹³ and R¹⁴ are each independently hydrogen or C₁-C₄ alkyl. In some embodiments, R³ is —(CH₂)ₘNR¹³S(O)₂R¹⁴, wherein m is 0, R¹³ is hydrogen, and R¹⁴ is C₁-C₄ alkyl such as methyl, ethyl, n-propyl, or isopropyl. In some embodiments, R³ is

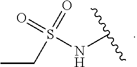 [SMILES: *NS(=O)(=O)CC]

In some embodiments, when R³ is —(CH₂)ₘNR¹³S(O)₂R¹⁴, then R² is 5- to 10-membered heteroaryl optionally substituted by R¹². In some embodiments, R³ is C₁-C₄ alkyl substituted by halogen, oxo, —CN or —OH. In some embodiments of a compound of Formula (I), R³ is C₁-C₄ alkyl substituted by —OH. In some embodiments of a compound of Formula (I), R³ is

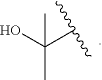 [SMILES: *C(C)(C)O]

In some embodiments, R³ is C₃-C₆ cycloalkyl optionally substituted by halogen, oxo, —CN, or —OH. In some embodiments, R³ is C₃-C₆ cycloalkyl which is unsubstituted. In some embodiments, R³ is

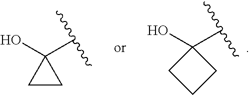 [SMILES: *C1(O)CC1 ; *C1(O)CCC1]

In some embodiments of a compound of Formula (I), R³ is

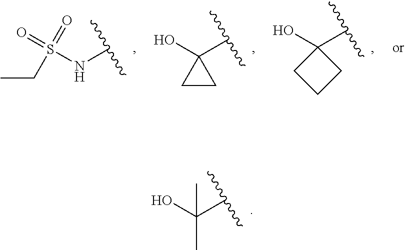

In some embodiments of a compound of Formula (I), R³ is

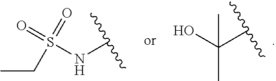

In some embodiments of a compound of Formula (I) or any related formula, or a tautomer or isomer thereof, or a pharmaceutically acceptable salt of any of the foregoing, R³ is C₁-C₄ alkyl substituted by halogen, oxo, —CN or —OH; and R² is hydrogen. In some embodiments, R³ is C₁-C₄ alkyl substituted by halogen, oxo, —CN or —OH; and R² is hydrogen, —C(O)NR¹⁰R¹¹, or 5- to 10-membered heteroaryl, wherein the —C(O)NR¹⁰R¹¹ and 5-to 10-membered heteroaryl are each optionally substituted by R¹². In some embodiments, R³ is

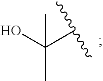 [SMILES: *C(C)(C)O]

and R² is hydrogen. In some embodiments, R³ is C₁-C₄ alkyl substituted by halogen, oxo, —CN or —OH; and R² is —C(O)NR¹⁰R¹¹ or 5- to 10-membered heteroaryl, wherein the —C(O)NR¹⁰R¹¹ and 5- to 10-membered heteroaryl are each optionally substituted by R¹². In some embodiments, R³

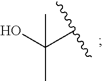 [SMILES: *C(C)(C)O]

and R² is —C(O)NR¹⁰R¹¹ or 5- to 10-membered heteroaryl, wherein the —C(O)NR¹⁰R¹¹ and 5- to 10-membered heteroaryl are each optionally substituted by R¹².

In some embodiments of a compound of Formula (I) or any related formula, or a tautomer or isomer thereof, or a pharmaceutically acceptable salt of any of the foregoing, W₁ is O and Z₁ is C—O—R^{c}. In some embodiments, W₁ is —NR^{w1}— and Z₁ is C—NR^{w1}—R^{c}. In some embodiments, R^{w1} is hydrogen, C₃-C₆ cycloalkyl or C₁-C₄ alkyl optionally substituted by oxo, OH or halogen. In some embodiments, R^{w1} is hydrogen. In some embodiments, R^{w1} is C₃-C₆ cycloalkyl. In some embodiments, R^{w1} is C₁-C₄ alkyl optionally substituted by oxo, OH or halogen. In some embodiments, R^{w1} is methyl. In some embodiments, W₁ is —NH— and Z₁ is C—NH—R^{c}.

In some embodiments of a compound of Formula (I) or any related formula, or a tautomer or isomer thereof, or a pharmaceutically acceptable salt of any of the foregoing, R^{c} is C₃_C₆ cycloalkyl, C₆-C₁₄ aryl, or 5- or 6-membered heteroaryl, wherein the C₃-C₆ cycloalkyl, C₆-C₁₄ aryl, and 5- or 6-membered heteroaryl of R^{c} are each independently optionally substituted by R^{c1}. In some embodiments, each R^{c1} is independently halogen or C₁-C₄ alkyl. In some embodiments of a compound of Formula (I), R^{c} is C₆-C₁₄ aryl optionally substituted by R^{c1}. In some embodiments of a compound of Formula (I), R^{c} is C₆-C₁₄ aryl which is unsubstituted. In some embodiments of a compound of Formula (I), R^{c} is phenyl optionally substituted by R^{c1}. In some embodiments, R^{c} is phenyl which is unsubstituted. In some embodiments, R^{c} is phenyl optionally substituted by R^{c1}, wherein each R^{c1} is independently halogen or C₁-C₄ alkyl. In some embodiments, R^{c} is phenyl optionally substituted by R^{c1}, wherein each R^{c1} is independently methyl or fluoro. In some embodiments of a compound of Formula (I), R^{c} is 5- or 6-membered heteroaryl optionally substituted by R^{c1}. In some embodiments, R^{c} is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is unsubstituted. In some embodiments, R^{c} is 5- or 6-membered heteroaryl optionally substituted by R^{c1}, such as pyridinyl, pyrazinyl, pyridazinyl, primidinyl, triazinyl, pyrrolyl, pyrazolyl, imidazolyl, triazolyl, tetrazolyl, oxazolyl, thiazolyl, thiazolyl, or furanyl, each of which is independently optionally substituted by R^{c1}. In some embodiments, R^{c} is C₃-C₆ cycloalkyl optionally substituted by R^{c1}. In some embodiments, R^{c} is phenyl or pyridinyl, each of which is independently optionally substituted by R^{c1}. In some embodiments, R^{c} is phenyl, pyridinyl, or cyclohexyl, each of which is independently optionally substituted by R^{c1}.

In some embodiments of a compound of Formula (I) or any related formula, or a tautomer or isomer thereof, or a pharmaceutically acceptable salt of any of the foregoing, R^{c} is

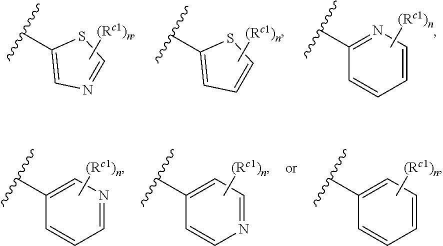

and n is 0, 1, 2, 3, 4, or 5, as permitted by the valence. In some embodiments, R^{c} is

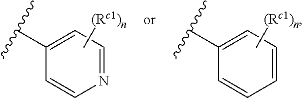

wherein n is 0, 1, 2, 3, or 4. In some embodiments, R^{c} is

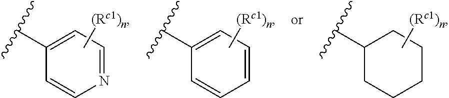

wherein n is 0, 1, 2, 3, or 4. In some embodiments, R^{c} is

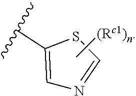

In some embodiments, R^{c} is

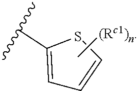

In some embodiments, R^{c} is

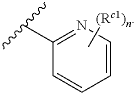

In some embodiments, R^{c} is

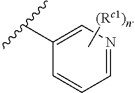

In some embodiments, R^{c} is

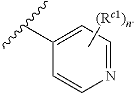

In some embodiments, R^{c} is

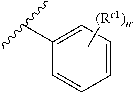

In some embodiments, each R^{c1} is independently halogen or C₁-C₄ alkyl.

In some embodiments of a compound of Formula (I) or any related formula, or a tautomer or isomer thereof, or a pharmaceutically acceptable salt of any of the foregoing, R^{c} is selected from the group consisting of:

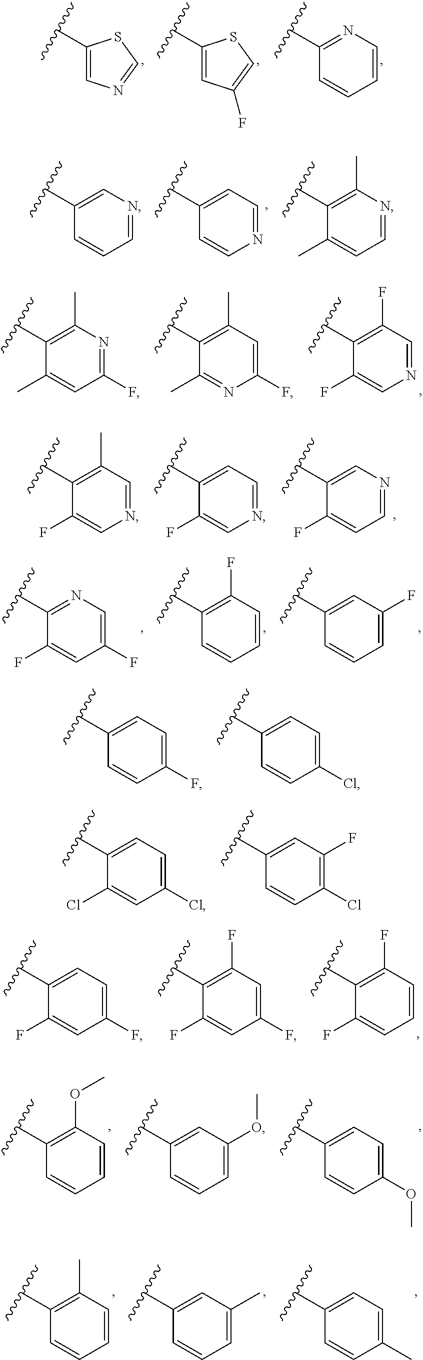

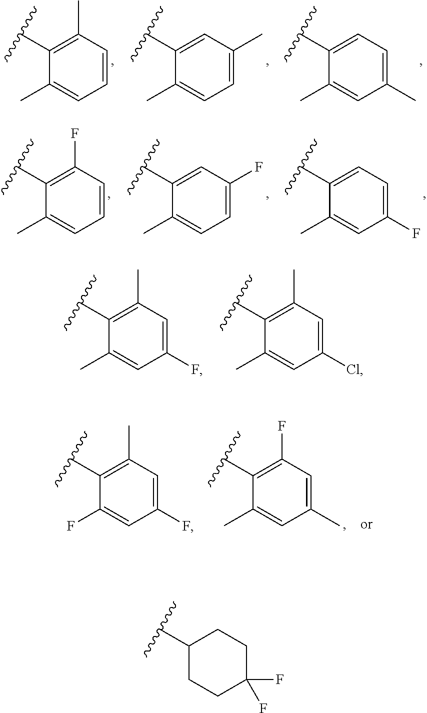

wherein the wavy lines denote attachment points. In some embodiments, R^{c} is

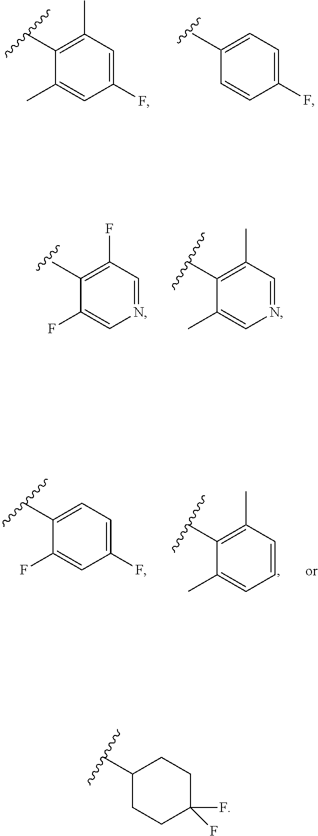

In some embodiments, R^{c} is

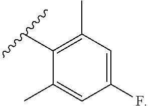 [SMILES: *c1c(C)cc(F)cc1C]

In some embodiments, R^{c} is

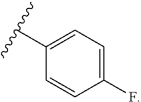 [SMILES: *c1ccc(F)cc1]

In some embodiments, R^{c} is

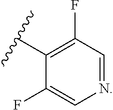 [SMILES: *c1c(F)cncc1F]

In some embodiments, R^{c} is

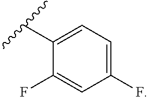 [SMILES: *c1ccc(F)cc1F]

In some embodiments, R^{c} is

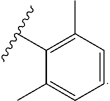 [SMILES: *c1c(C)cccc1C]

In some embodiments, R^{c} is

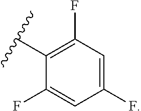 [SMILES: *c1c(F)cc(F)cc1F]

In some embodiments, R^{c} is

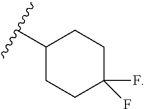 [SMILES: *C1CCC(F)(F)CC1]

In some embodiments of a compound of Formula (I) or any related formula, or a tautomer or isomer thereof, or a pharmaceutically acceptable salt of any of the foregoing, Z₂ is N. In some embodiments, Z₂ is C—W₂—R^{d}. In some embodiments, W₂ is —O—. In some embodiments, W₂ is a bond. In some embodiments, W₂ is —NR^{w2}—. In some embodiments, R^{w2} is hydrogen or C₁-C₄ alkyl optionally substituted by oxo, OH or halogen. In some embodiments, W₂ is —NR^{w2}— and R^{w2} is hydrogen. In some embodiments, R^{w2} is C₁-C₄ alkyl optionally substituted by oxo, OH or halogen. In some embodiments, R^{w2} is methyl. In some embodiments, R^{w2} is C₃-C₆ cycloalkyl. In some embodiments, W₂ is —NR^{w2}— and R^{w2} is —CH₃—. In some embodiments, W₂ is —NR^{w2}— and R^{w2} is hydrogen.

In some embodiments of a compound of Formula (I) or any related formula, or a tautomer or isomer thereof, or a pharmaceutically acceptable salt of any of the foregoing, R^{d} is hydrogen. In some embodiments, R^{d} is C₁-C₄ alkyl, such as methyl, ethyl, n-propyl, or isopropyl. In some embodiments, R^{d} is methyl. In some embodiments, Z₂ is C—W₂—R^{d}, wherein W₂ is a bond and R^{d} is hydrogen.

In some embodiments of a compound of Formula (I) or any related formula, or a tautomer or isomer thereof, or a pharmaceutically acceptable salt of any of the foregoing, Z₃ is N. In some embodiments, Z₃ is C—R^{e}. In some embodiments, R^{e} is hydrogen. In some embodiments, R^{e} is halogen, such as fluoro, chloro, bromo, or iodo. In some embodiments of a compound of Formula (I), R^{e} is cyano. In some embodiments, R^{e} is 3- to 6-membered heterocyclyl, such as tetrahydrofuranyl, pyrrolidinyl, piperidinyl, piperazinyl, morpholinyl, or thiomorpholinyl. In some embodiments, R^{e} is C₁-C₄ alkyl such as methyl, ethyl, n-propyl, or isopropyl. In some embodiments, Z₃ is C—R^{e} and R^{e} is hydrogen.

In some embodiments of a compound of Formula (I) or any related formula, or a tautomer or isomer thereof, or a pharmaceutically acceptable salt of any of the foregoing, Z₁ is C—O—R^{c}; Z₂ is C—W₂—R^{d}, wherein W² is a bond and R^{d} is hydrogen; and Z₃ is C—R^{e} and R^{e} is hydrogen.

It is to be understood that any variable for a compound of Formula (I) or any related formula may be combined with any other variable for a compound of Formula (I) or any related formulae the same as if each and every combination of variables were specifically and individually listed. For example, in some embodiments, X is O; R¹ is C₁-C₃ alkyl; G₁ is CR^{a}, wherein R^{a} is hydrogen; Z₂ is CH; Z₃ is CH; R³ is —(CH₂)ₘNR¹³S(O)₂R¹⁴; and R² is 5- to 10-membered heteroaryl optionally substituted by R¹². As another example, in some embodiments, X is O; R¹ is C₁-C₃ alkyl; G₁ is CR^{a}, wherein R^{a} is hydrogen; Z₂ is CH; Z₃ is CH; R³ is C₁-C₄ alkyl substituted by halogen, oxo, —CN; and R² is 5- to 10-membered heteroaryl optionally substituted by R¹².

In some embodiments of a compound of Formula (I) or any related formula where applicable, the compound has one or more of the following features:

### (I) X is O;

(II) R¹ is C₁-C₃ alkyl, such as methyl;
(III) G₁ is CR^{a}, wherein R^{a} is hydrogen;

### (IV) Z₂ is CH;

### (V) Z₃ is CH;

### (VI) R² is

(1) —C(O)NR¹⁰R¹¹ optionally substituted by R¹², such as

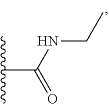 [SMILES: *C(=O)NCC]

(2) 5- to 10-membered heteroaryl optionally substituted by R¹², such as

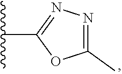 [SMILES: *c1nnc(C)o1]

or

(3) hydrogen;

### (VII) R³ is

(4) —(CH₂)ₘNR¹³S(O)₂R¹⁴, such as

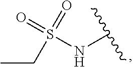 [SMILES: *NS(=O)(=O)CC]

or

(5) C₁-C₄ alkyl substituted by —OH, such as

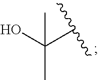 [SMILES: *C(C)(C)O]

and
(VIII) Z₁ is C—O—R^{c}, wherein R^{c} is phenyl, pyridinyl, or cyclohexyl, each of which is independently optionally substituted by R^{c1}, such as

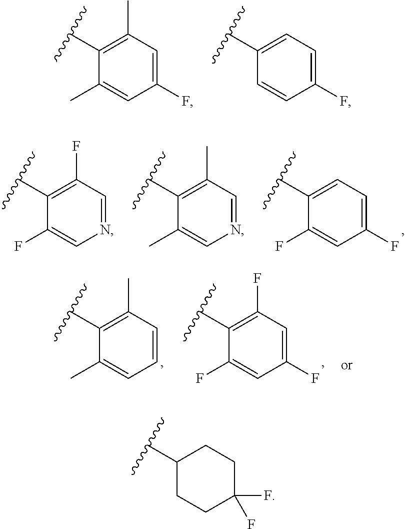

In some embodiments, (I) applies. In some embodiments, (II) applies. In some embodiments, (III) applies. In some embodiments, (IV) applies. In some embodiments, (V) applies. In some embodiments, (VI) applies. In some embodiments, (VII) applies. In some embodiments, (VIII) applies. In some embodiments, (1) applies. In some embodiments, (2) applies. In some embodiments, (3) applies. In some embodiments, (4) applies. In some embodiments, (5) applies. In some embodiments, (I), (II), (III), (IV), (V), (VI), (VII), and (VIII) apply. In some embodiments, (I), (II), (III), (IV), (V), and (1) apply. In some embodiments, (I), (II), (III), (IV), (V), and (4) apply. In some embodiments, (I), (II), (III), (IV), (V), and (5) apply. In some embodiments, (I), (II), (III), (IV), (V), and (VIII) apply. In some embodiments, (I), (II), (III), (IV), (V), and (2) apply. In some embodiments, (I), (II), (III), (IV), (V), and (3) apply. In some embodiments, (I), (II), (III), (IV), (V), (1), (VII), and (VIII) apply. In some embodiments, (I), (II), (III), (IV), (V), (2), (VII), and (VIII) apply. In some embodiments, (I), (II), (III), (IV), (V), (3), (VII), and (VIII) apply. In some embodiments, (I), (II), (III), (IV), (V), (VI), (4), and (VIII) apply. In some embodiments, (I), (II), (III), (IV), (V), (VI), (5), and (VIII) apply. In some embodiments, (4) and (VIII) apply. In some embodiments, (5) and (VIII) apply. In some embodiments, (1) and (VIII) apply. In some embodiments, (2) and (VIII) apply. In some embodiments, (3) and (VIII) apply. In some embodiments, (1) and (4) apply. In some embodiments, (1) and (5) apply. In some embodiments, (2) and (4) apply. In some embodiments, (2) and (5) apply. In some embodiments, (3) and (4) apply. In some embodiments, (3) and (5) apply. In some embodiments, (I), (II), (III), (IV), (V), (2), (4), and (VIII) apply. In some embodiments, (I), (II), (III), (IV), (V), (2), (5), and (VIII) apply. In some embodiments, (I), (II), (III), (IV), (V), (3), (5), and (VIII) apply.

In some embodiments of a compound of Formula (I) or any related formula where applicable, the compound has one or more of the following features:

### (I) X is O;

(II) R¹ is C₁-C₃ alkyl, such as methyl;
(III) G₁ is CR^{a}, wherein R^{a} is hydrogen;

### (IV) Z₂ is N;

### (V) Z₃ is CH;

### (VI) R² is

(1) —C(O)NR¹⁰R¹¹ optionally substituted by R¹², such as

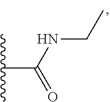 [SMILES: *C(=O)NCC]

(2) 5- to 10-membered heteroaryl optionally substituted by R¹², such as

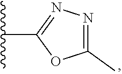 [SMILES: *c1nnc(C)o1]

or

(3) hydrogen;

### (VII) R³ is

(4) —(CH₂)ₘNR¹³S(O)₂R¹⁴, such as

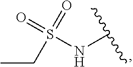 [SMILES: *NS(=O)(=O)CC]

or

(5) C₁-C₄ alkyl substituted by —OH, such as

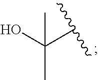 [SMILES: *C(C)(C)O]

and
(VIII) Z₁ is C—O—R^{c}, wherein R^{c} is phenyl, pyridinyl, or cyclohexyl, each of which is independently optionally substituted by R^{c1}, such as

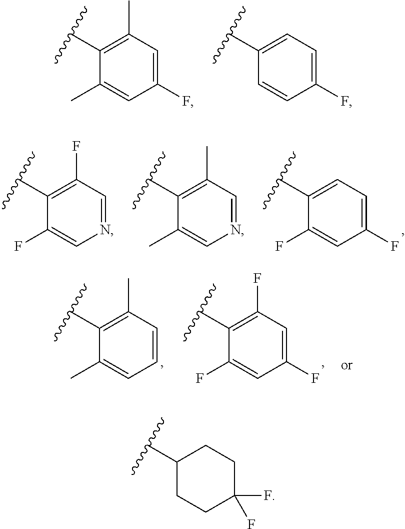

In some embodiments, (I) applies. In some embodiments, (II) applies. In some embodiments, (III) applies. In some embodiments, (IV) applies. In some embodiments, (V) applies. In some embodiments, (VI) applies. In some embodiments, (VII) applies. In some embodiments, (VIII) applies. In some embodiments, (1) applies. In some embodiments, (2) applies. In some embodiments, (3) applies. In some embodiments, (4) applies. In some embodiments, (5) applies. In some embodiments, (I), (II), (III), (IV), (V), (VI), (VII), and (VIII) apply. In some embodiments, (I), (II), (III), (IV), (V), and (1) apply. In some embodiments, (I), (II), (III), (IV), (V), and (4) apply. In some embodiments, (I), (II), (III), (IV), (V), and (5) apply. In some embodiments, (I), (II), (III), (IV), (V), and (VIII) apply. In some embodiments, (I), (II), (III), (IV), (V), and (2) apply. In some embodiments, (I), (II), (III), (IV), (V), and (3) apply. In some embodiments, (I), (II), (III), (IV), (V), (1), (VII), and (VIII) apply. In some embodiments, (I), (II), (III), (IV), (V), (2), (VII), and (VIII) apply. In some embodiments, (I), (II), (III), (IV), (V), (3), (VII), and (VIII) apply. In some embodiments, (I), (II), (III), (IV), (V), (VI), (4), and (VIII) apply. In some embodiments, (I), (II), (III), (IV), (V), (VI), (5), and (VIII) apply. In some embodiments, (4) and (VIII) apply. In some embodiments, (5) and (VIII) apply. In some embodiments, (1) and (VIII) apply. In some embodiments, (2) and (VIII) apply. In some embodiments, (3) and (VIII) apply. In some embodiments, (1) and (4) apply. In some embodiments, (1) and (5) apply. In some embodiments, (2) and (4) apply. In some embodiments, (2) and (5) apply. In some embodiments, (3) and (4) apply. In some embodiments, (3) and (5) apply. In some embodiments, (I), (II), (III), (IV), (V), (2), (4), and (VIII) apply. In some embodiments, (I), (II), (III), (IV), (V), (2), (5), and (VIII) apply. In some embodiments, (I), (II), (III), (IV), (V), (3), (5), and (VIII) apply.

Also provided are salts of compounds referred to herein, such as pharmaceutically acceptable salts. The disclosure also includes any or all of the stereochemical forms, including any enantiomeric or diastereomeric forms, and any tautomers or other forms of the compounds described.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds are listed in Table 1.

TABLE 1

| No. | Structure |
|---|---|
| 1 | 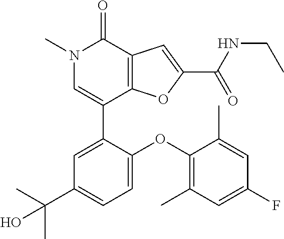 [SMILES: CCNC(=O)c1cc2c(=O)n(C)cc(-c3cc(C(C)(C)O)ccc3Oc3c(C)cc(F)cc3C)c2o1] |
| 2 | 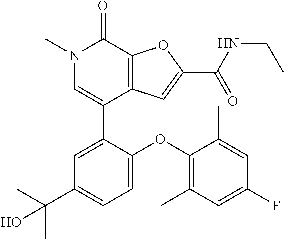 [SMILES: CCNC(=O)c1cc2c(-c3cc(C(C)(C)O)ccc3Oc3c(C)cc(F)cc3C)cn(C)c(=O)c2o1] |
| 3 | 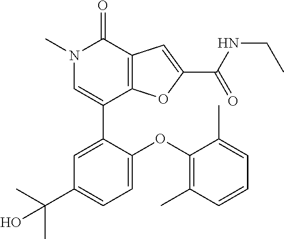 [SMILES: CCNC(=O)c1cc2c(=O)n(C)cc(-c3cc(C(C)(C)O)ccc3Oc3c(C)cccc3C)c2o1] |
| 4 | 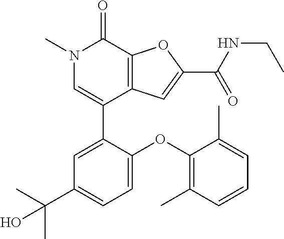 [SMILES: CCNC(=O)c1cc2c(-c3cc(C(C)(C)O)ccc3Oc3c(C)cccc3C)cn(C)c(=O)c2o1] |
| 5 | 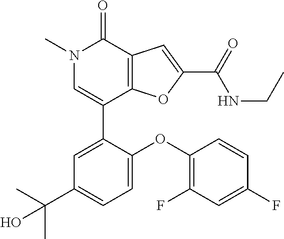 [SMILES: CCNC(=O)c1cc2c(=O)n(C)cc(-c3cc(C(C)(C)O)ccc3Oc3ccc(F)cc3F)c2o1] |
| 6 | 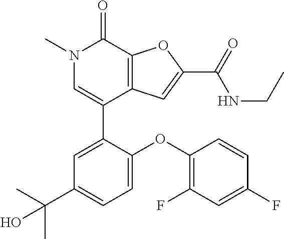 [SMILES: CCNC(=O)c1cc2c(-c3cc(C(C)(C)O)ccc3Oc3ccc(F)cc3F)cn(C)c(=O)c2o1] |
| 7 | 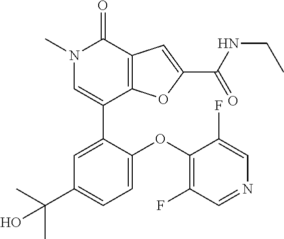 [SMILES: CCNC(=O)c1cc2c(=O)n(C)cc(-c3cc(C(C)(C)O)ccc3Oc3c(F)cncc3F)c2o1] |
| 8 | 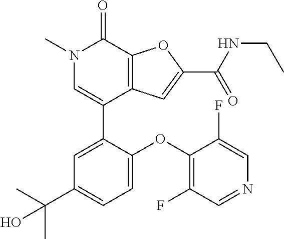 [SMILES: CCNC(=O)c1cc2c(-c3cc(C(C)(C)O)ccc3Oc3c(F)cncc3F)cn(C)c(=O)c2o1] |
| 9 | 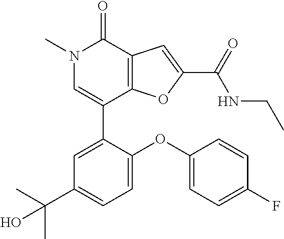 [SMILES: CCNC(=O)c1cc2c(=O)n(C)cc(-c3cc(C(C)(C)O)ccc3Oc3ccc(F)cc3)c2o1] |
| 10 | 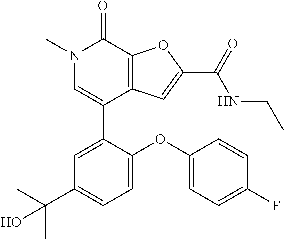 [SMILES: CCNC(=O)c1cc2c(-c3cc(C(C)(C)O)ccc3Oc3ccc(F)cc3)cn(C)c(=O)c2o1] |
| 11 | 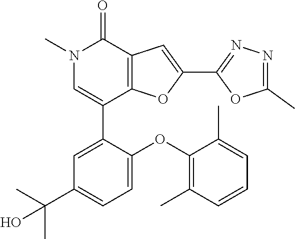 [SMILES: Cc1nnc(-c2cc3c(=O)n(C)cc(-c4cc(C(C)(C)O)ccc4Oc4c(C)cccc4C)c3o2)o1] |
| 12 | 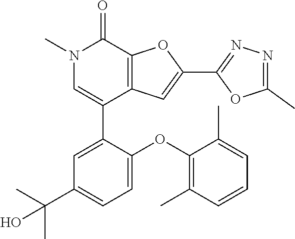 [SMILES: Cc1nnc(-c2cc3c(-c4cc(C(C)(C)O)ccc4Oc4c(C)cccc4C)cn(C)c(=O)c3o2)o1] |
| 13 | 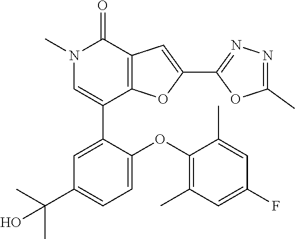 [SMILES: Cc1nnc(-c2cc3c(=O)n(C)cc(-c4cc(C(C)(C)O)ccc4Oc4c(C)cc(F)cc4C)c3o2)o1] |
| 14 | 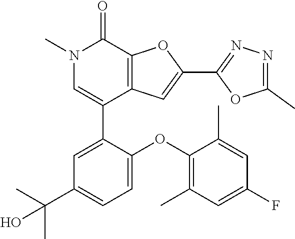 [SMILES: Cc1nnc(-c2cc3c(-c4cc(C(C)(C)O)ccc4Oc4c(C)cc(F)cc4C)cn(C)c(=O)c3o2)o1] |
| 15 | 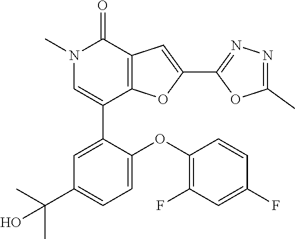 [SMILES: Cc1nnc(-c2cc3c(=O)n(C)cc(-c4cc(C(C)(C)O)ccc4Oc4ccc(F)cc4F)c3o2)o1] |
| 16 | 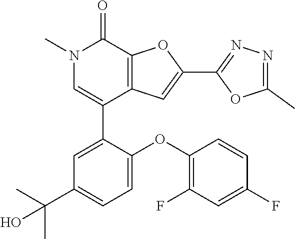 [SMILES: Cc1nnc(-c2cc3c(-c4cc(C(C)(C)O)ccc4Oc4ccc(F)cc4F)cn(C)c(=O)c3o2)o1] |
| 17 | 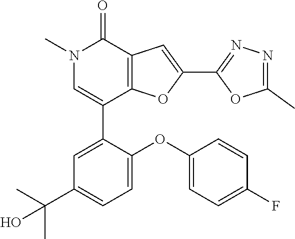 [SMILES: Cc1nnc(-c2cc3c(=O)n(C)cc(-c4cc(C(C)(C)O)ccc4Oc4ccc(F)cc4)c3o2)o1] |
| 18 | 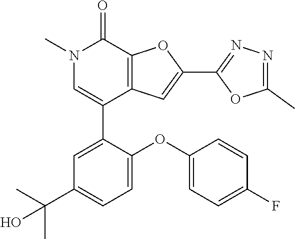 [SMILES: Cc1nnc(-c2cc3c(-c4cc(C(C)(C)O)ccc4Oc4ccc(F)cc4)cn(C)c(=O)c3o2)o1] |
| 19 | 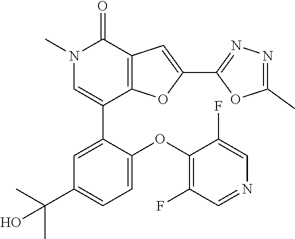 [SMILES: Cc1nnc(-c2cc3c(=O)n(C)cc(-c4cc(C(C)(C)O)ccc4Oc4c(F)cncc4F)c3o2)o1] |
| 20 | 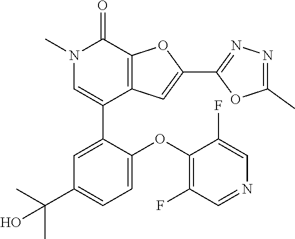 [SMILES: Cc1nnc(-c2cc3c(-c4cc(C(C)(C)O)ccc4Oc4c(F)cncc4F)cn(C)c(=O)c3o2)o1] |
| 21 | 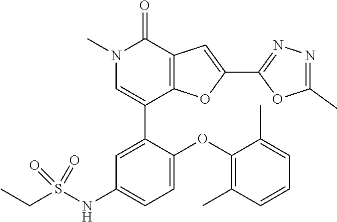 [SMILES: CCS(=O)(=O)Nc1ccc(Oc2c(C)cccc2C)c(-c2cn(C)c(=O)c3cc(-c4nnc(C)o4)oc23)c1] |
| 22 | 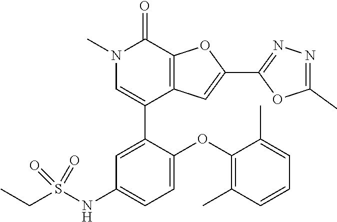 [SMILES: CCS(=O)(=O)Nc1ccc(Oc2c(C)cccc2C)c(-c2cn(C)c(=O)c3oc(-c4nnc(C)o4)cc23)c1] |
| 23 | 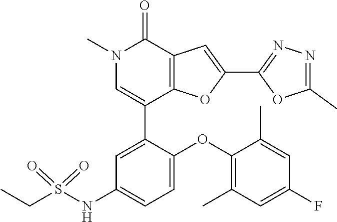 [SMILES: CCS(=O)(=O)Nc1ccc(Oc2c(C)cc(F)cc2C)c(-c2cn(C)c(=O)c3cc(-c4nnc(C)o4)oc23)c1] |
| 24 | 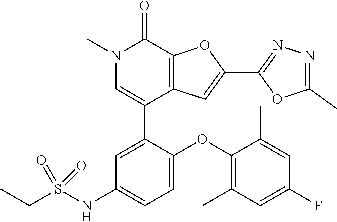 [SMILES: CCS(=O)(=O)Nc1ccc(Oc2c(C)cc(F)cc2C)c(-c2cn(C)c(=O)c3oc(-c4nnc(C)o4)cc23)c1] |
| 25 | 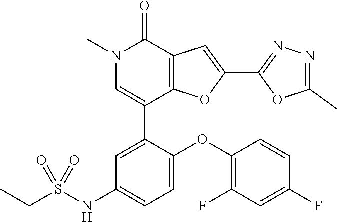 [SMILES: CCS(=O)(=O)Nc1ccc(Oc2ccc(F)cc2F)c(-c2cn(C)c(=O)c3cc(-c4nnc(C)o4)oc23)c1] |
| 26 | 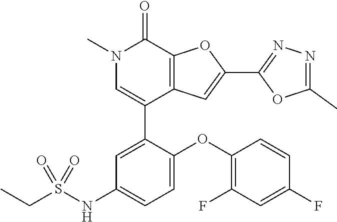 [SMILES: CCS(=O)(=O)Nc1ccc(Oc2ccc(F)cc2F)c(-c2cn(C)c(=O)c3oc(-c4nnc(C)o4)cc23)c1] |
| 27 | 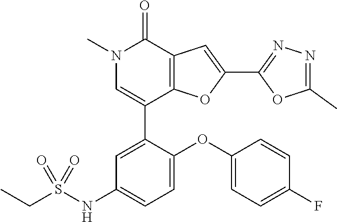 [SMILES: CCS(=O)(=O)Nc1ccc(Oc2ccc(F)cc2)c(-c2cn(C)c(=O)c3cc(-c4nnc(C)o4)oc23)c1] |
| 28 | 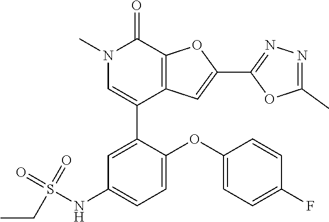 [SMILES: CCS(=O)(=O)Nc1ccc(Oc2ccc(F)cc2)c(-c2cn(C)c(=O)c3oc(-c4nnc(C)o4)cc23)c1] |
| 29 | 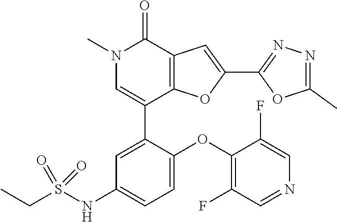 [SMILES: CCS(=O)(=O)Nc1ccc(Oc2c(F)cncc2F)c(-c2cn(C)c(=O)c3cc(-c4nnc(C)o4)oc23)c1] |
| 30 | 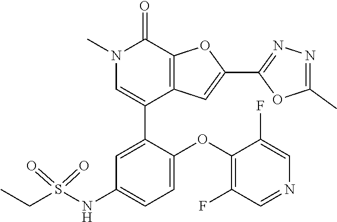 [SMILES: CCS(=O)(=O)Nc1ccc(Oc2c(F)cncc2F)c(-c2cn(C)c(=O)c3oc(-c4nnc(C)o4)cc23)c1] |
| 31 | 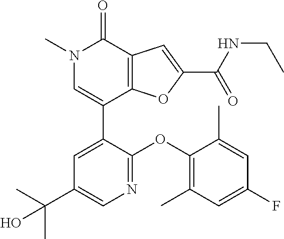 [SMILES: CCNC(=O)c1cc2c(=O)n(C)cc(-c3cc(C(C)(C)O)cnc3Oc3c(C)cc(F)cc3C)c2o1] |
| 32 | 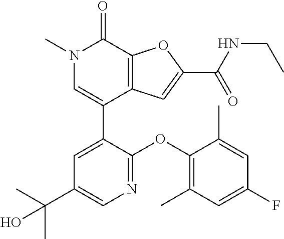 [SMILES: CCNC(=O)c1cc2c(-c3cc(C(C)(C)O)cnc3Oc3c(C)cc(F)cc3C)cn(C)c(=O)c2o1] |
| 33 | 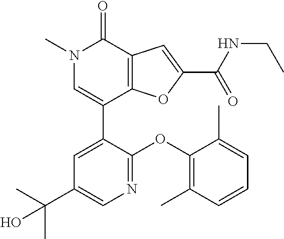 [SMILES: CCNC(=O)c1cc2c(=O)n(C)cc(-c3cc(C(C)(C)O)cnc3Oc3c(C)cccc3C)c2o1] |
| 34 | 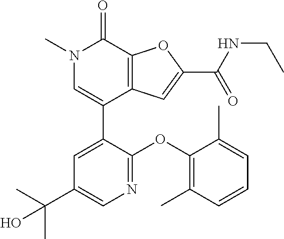 [SMILES: CCNC(=O)c1cc2c(-c3cc(C(C)(C)O)cnc3Oc3c(C)cccc3C)cn(C)c(=O)c2o1] |
| 35 | 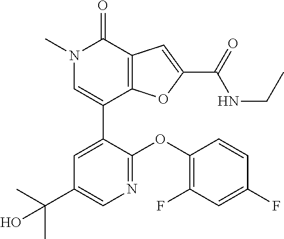 [SMILES: CCNC(=O)c1cc2c(=O)n(C)cc(-c3cc(C(C)(C)O)cnc3Oc3ccc(F)cc3F)c2o1] |
| 36 | 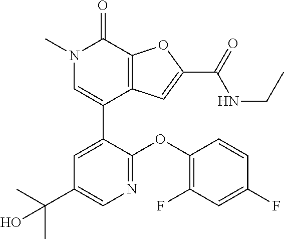 [SMILES: CCNC(=O)c1cc2c(-c3cc(C(C)(C)O)cnc3Oc3ccc(F)cc3F)cn(C)c(=O)c2o1] |
| 37 | 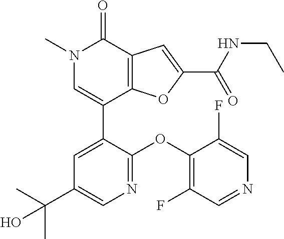 [SMILES: CCNC(=O)c1cc2c(=O)n(C)cc(-c3cc(C(C)(C)O)cnc3Oc3c(F)cncc3F)c2o1] |
| 38 | 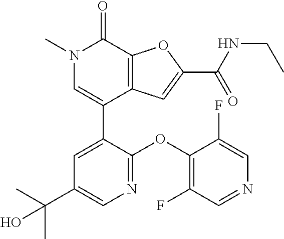 [SMILES: CCNC(=O)c1cc2c(-c3cc(C(C)(C)O)cnc3Oc3c(F)cncc3F)cn(C)c(=O)c2o1] |
| 39 | 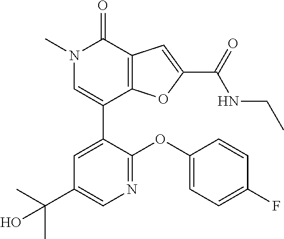 [SMILES: CCNC(=O)c1cc2c(=O)n(C)cc(-c3cc(C(C)(C)O)cnc3Oc3ccc(F)cc3)c2o1] |
| 40 | 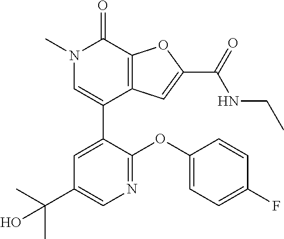 [SMILES: CCNC(=O)c1cc2c(-c3cc(C(C)(C)O)cnc3Oc3ccc(F)cc3)cn(C)c(=O)c2o1] |
| 41 | 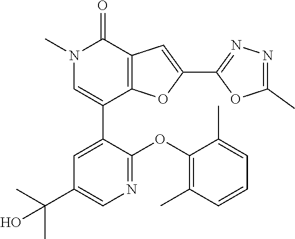 [SMILES: Cc1nnc(-c2cc3c(=O)n(C)cc(-c4cc(C(C)(C)O)cnc4Oc4c(C)cccc4C)c3o2)o1] |
| 42 | 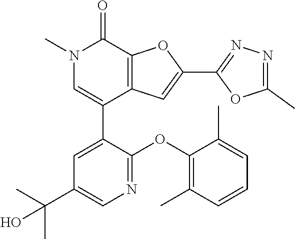 [SMILES: Cc1nnc(-c2cc3c(-c4cc(C(C)(C)O)cnc4Oc4c(C)cccc4C)cn(C)c(=O)c3o2)o1] |
| 43 | 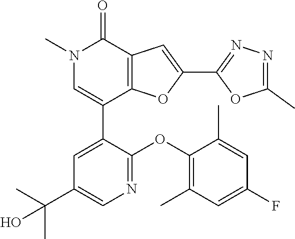 [SMILES: Cc1nnc(-c2cc3c(=O)n(C)cc(-c4cc(C(C)(C)O)cnc4Oc4c(C)cc(F)cc4C)c3o2)o1] |
| 44 | 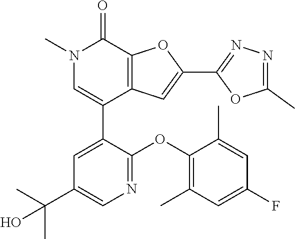 [SMILES: Cc1nnc(-c2cc3c(-c4cc(C(C)(C)O)cnc4Oc4c(C)cc(F)cc4C)cn(C)c(=O)c3o2)o1] |
| 45 | 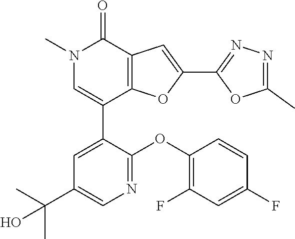 [SMILES: Cc1nnc(-c2cc3c(=O)n(C)cc(-c4cc(C(C)(C)O)cnc4Oc4ccc(F)cc4F)c3o2)o1] |
| 46 | 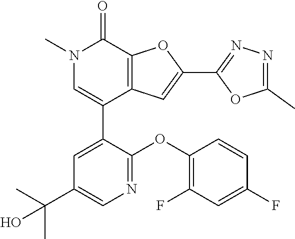 [SMILES: Cc1nnc(-c2cc3c(-c4cc(C(C)(C)O)cnc4Oc4ccc(F)cc4F)cn(C)c(=O)c3o2)o1] |
| 47 | 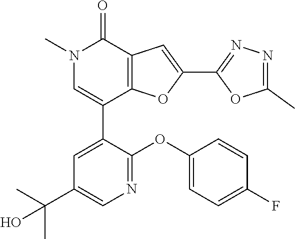 [SMILES: Cc1nnc(-c2cc3c(=O)n(C)cc(-c4cc(C(C)(C)O)cnc4Oc4ccc(F)cc4)c3o2)o1] |
| 48 | 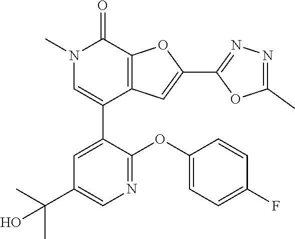 [SMILES: Cc1nnc(-c2cc3c(-c4cc(C(C)(C)O)cnc4Oc4ccc(F)cc4)cn(C)c(=O)c3o2)o1] |
| 49 | 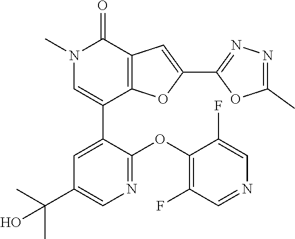 [SMILES: Cc1nnc(-c2cc3c(=O)n(C)cc(-c4cc(C(C)(C)O)cnc4Oc4c(F)cncc4F)c3o2)o1] |
| 50 | 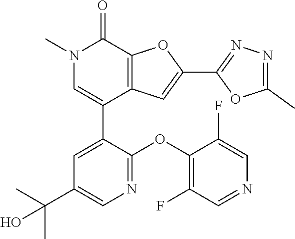 [SMILES: Cc1nnc(-c2cc3c(-c4cc(C(C)(C)O)cnc4Oc4c(F)cncc4F)cn(C)c(=O)c3o2)o1] |
| 51 | 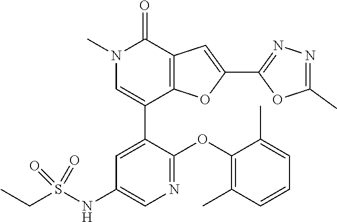 [SMILES: CCS(=O)(=O)Nc1cnc(Oc2c(C)cccc2C)c(-c2cn(C)c(=O)c3cc(-c4nnc(C)o4)oc23)c1] |
| 52 | 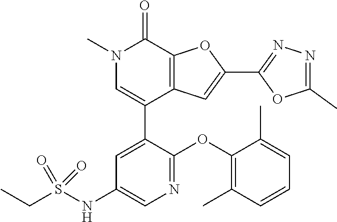 [SMILES: CCS(=O)(=O)Nc1cnc(Oc2c(C)cccc2C)c(-c2cn(C)c(=O)c3oc(-c4nnc(C)o4)cc23)c1] |
| 53 | 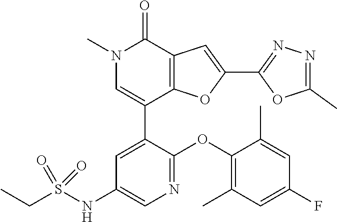 [SMILES: CCS(=O)(=O)Nc1cnc(Oc2c(C)cc(F)cc2C)c(-c2cn(C)c(=O)c3cc(-c4nnc(C)o4)oc23)c1] |
| 54 | 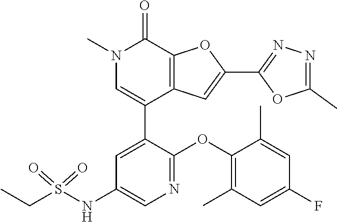 [SMILES: CCS(=O)(=O)Nc1cnc(Oc2c(C)cc(F)cc2C)c(-c2cn(C)c(=O)c3oc(-c4nnc(C)o4)cc23)c1] |
| 55 | 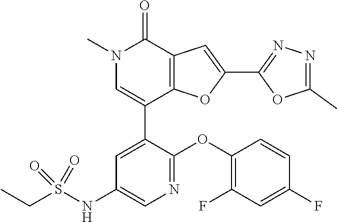 [SMILES: CCS(=O)(=O)Nc1cnc(Oc2ccc(F)cc2F)c(-c2cn(C)c(=O)c3cc(-c4nnc(C)o4)oc23)c1] |
| 56 | 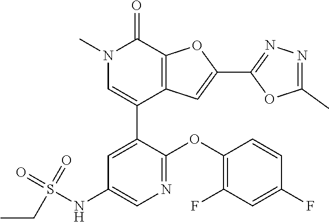 [SMILES: CCS(=O)(=O)Nc1cnc(Oc2ccc(F)cc2F)c(-c2cn(C)c(=O)c3oc(-c4nnc(C)o4)cc23)c1] |
| 57 | 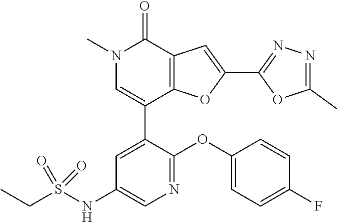 [SMILES: CCS(=O)(=O)Nc1cnc(Oc2ccc(F)cc2)c(-c2cn(C)c(=O)c3cc(-c4nnc(C)o4)oc23)c1] |
| 58 | 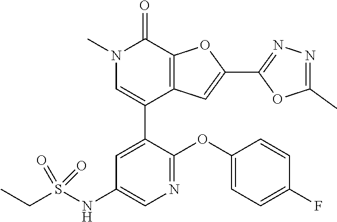 [SMILES: CCS(=O)(=O)Nc1cnc(Oc2ccc(F)cc2)c(-c2cn(C)c(=O)c3oc(-c4nnc(C)o4)cc23)c1] |
| 59 | 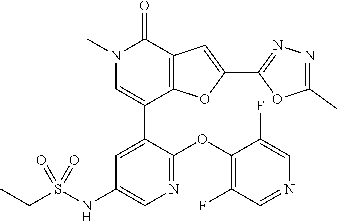 [SMILES: CCS(=O)(=O)Nc1cnc(Oc2c(F)cncc2F)c(-c2cn(C)c(=O)c3cc(-c4nnc(C)o4)oc23)c1] |
| 60 | 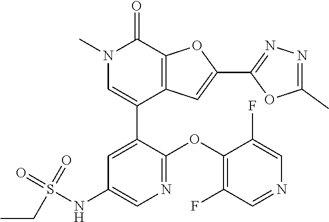 [SMILES: CCS(=O)(=O)Nc1cnc(Oc2c(F)cncc2F)c(-c2cn(C)c(=O)c3oc(-c4nnc(C)o4)cc23)c1] |
| 61 | 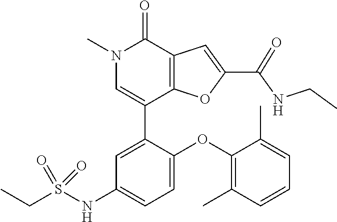 [SMILES: CCNC(=O)c1cc2c(=O)n(C)cc(-c3cc(NS(=O)(=O)CC)ccc3Oc3c(C)cccc3C)c2o1] |
| 62 | 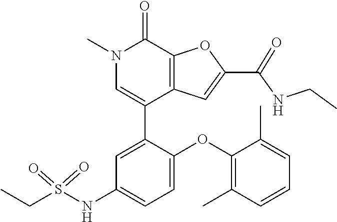 [SMILES: CCNC(=O)c1cc2c(-c3cc(NS(=O)(=O)CC)ccc3Oc3c(C)cccc3C)cn(C)c(=O)c2o1] |
| 63 | 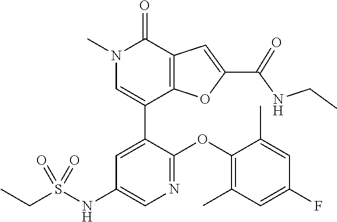 [SMILES: CCNC(=O)c1cc2c(=O)n(C)cc(-c3cc(NS(=O)(=O)CC)cnc3Oc3c(C)cc(F)cc3C)c2o1] |
| 64 | 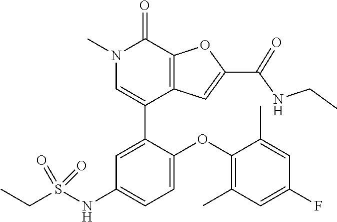 [SMILES: CCNC(=O)c1cc2c(-c3cc(NS(=O)(=O)CC)ccc3Oc3c(C)cc(F)cc3C)cn(C)c(=O)c2o1] |
| 65 | 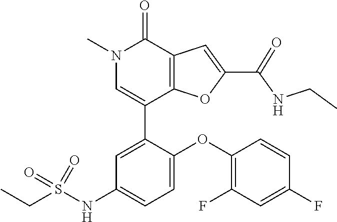 [SMILES: CCNC(=O)c1cc2c(=O)n(C)cc(-c3cc(NS(=O)(=O)CC)ccc3Oc3ccc(F)cc3F)c2o1] |
| 66 | 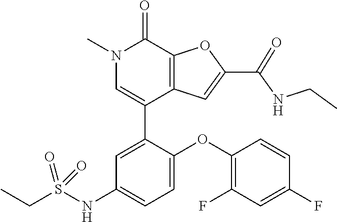 [SMILES: CCNC(=O)c1cc2c(-c3cc(NS(=O)(=O)CC)ccc3Oc3ccc(F)cc3F)cn(C)c(=O)c2o1] |
| 67 | 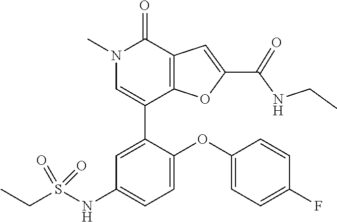 [SMILES: CCNC(=O)c1cc2c(=O)n(C)cc(-c3cc(NS(=O)(=O)CC)ccc3Oc3ccc(F)cc3)c2o1] |
| 68 | 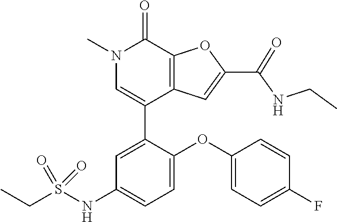 [SMILES: CCNC(=O)c1cc2c(-c3cc(NS(=O)(=O)CC)ccc3Oc3ccc(F)cc3)cn(C)c(=O)c2o1] |
| 69 | 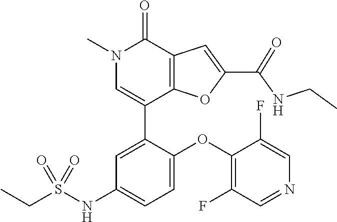 [SMILES: CCNC(=O)c1cc2c(=O)n(C)cc(-c3cc(NS(=O)(=O)CC)ccc3Oc3c(F)cncc3F)c2o1] |
| 70 | 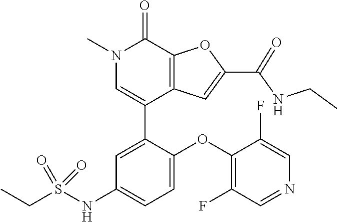 [SMILES: CCNC(=O)c1cc2c(-c3cc(NS(=O)(=O)CC)ccc3Oc3c(F)cncc3F)cn(C)c(=O)c2o1] |
| 71 | 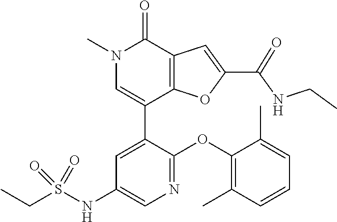 [SMILES: CCNC(=O)c1cc2c(=O)n(C)cc(-c3cc(NS(=O)(=O)CC)cnc3Oc3c(C)cccc3C)c2o1] |
| 72 | 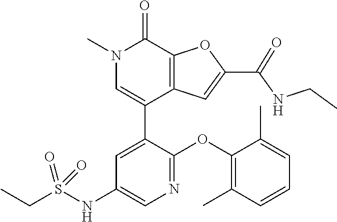 [SMILES: CCNC(=O)c1cc2c(-c3cc(NS(=O)(=O)CC)cnc3Oc3c(C)cccc3C)cn(C)c(=O)c2o1] |
| 73 | 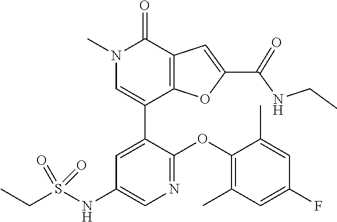 [SMILES: CCNC(=O)c1cc2c(=O)n(C)cc(-c3cc(NS(=O)(=O)CC)cnc3Oc3c(C)cc(F)cc3C)c2o1] |
| 74 | 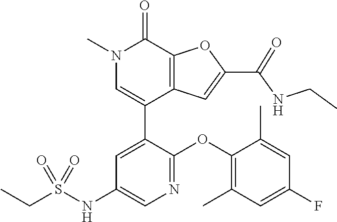 [SMILES: CCNC(=O)c1cc2c(-c3cc(NS(=O)(=O)CC)cnc3Oc3c(C)cc(F)cc3C)cn(C)c(=O)c2o1] |
| 75 | 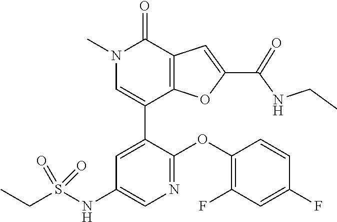 [SMILES: CCNC(=O)c1cc2c(=O)n(C)cc(-c3cc(NS(=O)(=O)CC)cnc3Oc3ccc(F)cc3F)c2o1] |
| 76 | 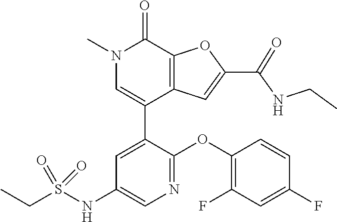 [SMILES: CCNC(=O)c1cc2c(-c3cc(NS(=O)(=O)CC)cnc3Oc3ccc(F)cc3F)cn(C)c(=O)c2o1] |
| 77 | 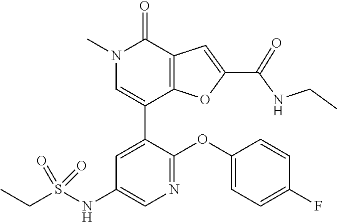 [SMILES: CCNC(=O)c1cc2c(=O)n(C)cc(-c3cc(NS(=O)(=O)CC)cnc3Oc3ccc(F)cc3)c2o1] |
| 78 | 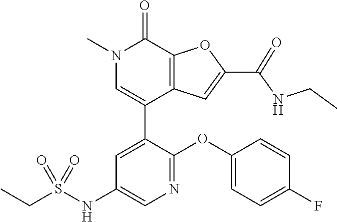 [SMILES: CCNC(=O)c1cc2c(-c3cc(NS(=O)(=O)CC)cnc3Oc3ccc(F)cc3)cn(C)c(=O)c2o1] |
| 79 | 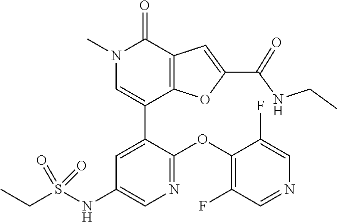 [SMILES: CCNC(=O)c1cc2c(=O)n(C)cc(-c3cc(NS(=O)(=O)CC)cnc3Oc3c(F)cncc3F)c2o1] |
| 80 | 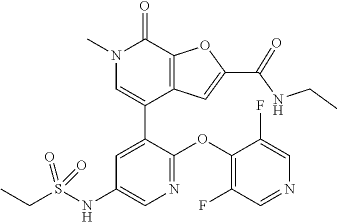 [SMILES: CCNC(=O)c1cc2c(-c3cc(NS(=O)(=O)CC)cnc3Oc3c(F)cncc3F)cn(C)c(=O)c2o1] |
| 81 | 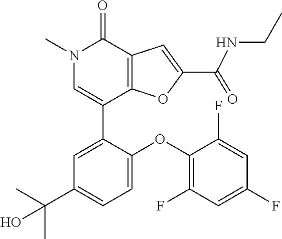 [SMILES: CCNC(=O)c1cc2c(=O)n(C)cc(-c3cc(C(C)(C)O)ccc3Oc3c(F)cc(F)cc3F)c2o1] |
| 82 | 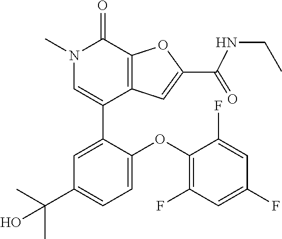 [SMILES: CCNC(=O)c1cc2c(-c3cc(C(C)(C)O)ccc3Oc3c(F)cc(F)cc3F)cn(C)c(=O)c2o1] |
| 83 | 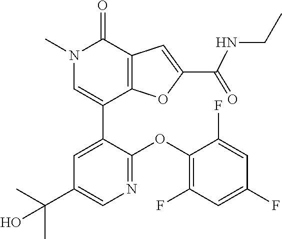 [SMILES: CCNC(=O)c1cc2c(=O)n(C)cc(-c3cc(C(C)(C)O)cnc3Oc3c(F)cc(F)cc3F)c2o1] |
| 84 | 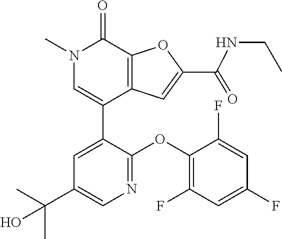 [SMILES: CCNC(=O)c1cc2c(-c3cc(C(C)(C)O)cnc3Oc3c(F)cc(F)cc3F)cn(C)c(=O)c2o1] |
| 85 | 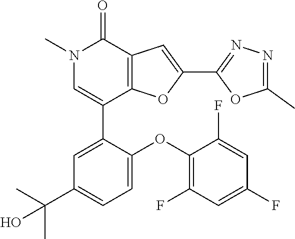 [SMILES: Cc1nnc(-c2cc3c(=O)n(C)cc(-c4cc(C(C)(C)O)ccc4Oc4c(F)cc(F)cc4F)c3o2)o1] |
| 86 | 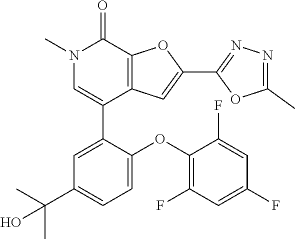 [SMILES: Cc1nnc(-c2cc3c(-c4cc(C(C)(C)O)ccc4Oc4c(F)cc(F)cc4F)cn(C)c(=O)c3o2)o1] |
| 87 | 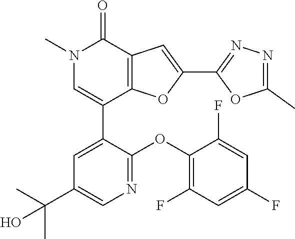 [SMILES: Cc1nnc(-c2cc3c(=O)n(C)cc(-c4cc(C(C)(C)O)cnc4Oc4c(F)cc(F)cc4F)c3o2)o1] |
| 88 | 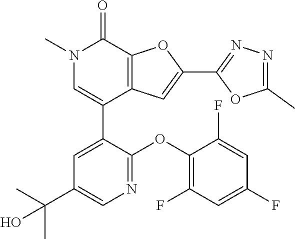 [SMILES: Cc1nnc(-c2cc3c(-c4cc(C(C)(C)O)cnc4Oc4c(F)cc(F)cc4F)cn(C)c(=O)c3o2)o1] |
| 89 | 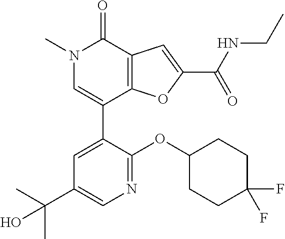 [SMILES: CCNC(=O)c1cc2c(=O)n(C)cc(-c3cc(C(C)(C)O)cnc3OC3CCC(F)(F)CC3)c2o1] |
| 90 | 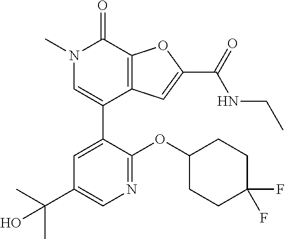 [SMILES: CCNC(=O)c1cc2c(-c3cc(C(C)(C)O)cnc3OC3CCC(F)(F)CC3)cn(C)c(=O)c2o1] |
| 91 | 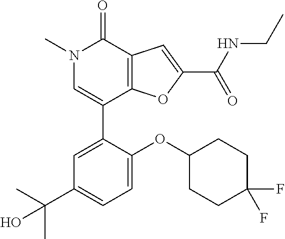 [SMILES: CCNC(=O)c1cc2c(=O)n(C)cc(-c3cc(C(C)(C)O)ccc3OC3CCC(F)(F)CC3)c2o1] |
| 92 | 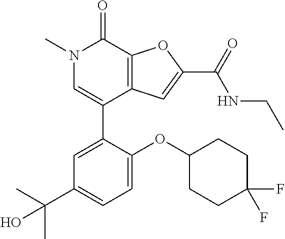 [SMILES: CCNC(=O)c1cc2c(-c3cc(C(C)(C)O)ccc3OC3CCC(F)(F)CC3)cn(C)c(=O)c2o1] |
| 93 | 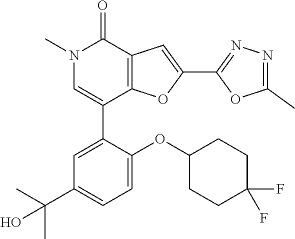 [SMILES: Cc1nnc(-c2cc3c(=O)n(C)cc(-c4cc(C(C)(C)O)ccc4OC4CCC(F)(F)CC4)c3o2)o1] |
| 94 | 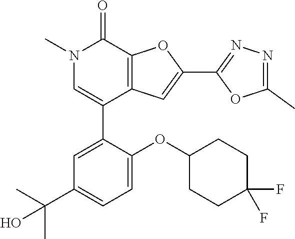 [SMILES: Cc1nnc(-c2cc3c(-c4cc(C(C)(C)O)ccc4OC4CCC(F)(F)CC4)cn(C)c(=O)c3o2)o1] |
| 95 | 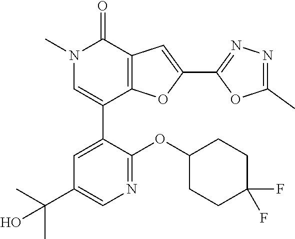 [SMILES: Cc1nnc(-c2cc3c(=O)n(C)cc(-c4cc(C(C)(C)O)cnc4OC4CCC(F)(F)CC4)c3o2)o1] |
| 96 | 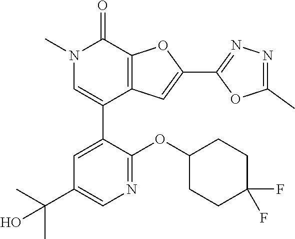 [SMILES: Cc1nnc(-c2cc3c(-c4cc(C(C)(C)O)cnc4OC4CCC(F)(F)CC4)cn(C)c(=O)c3o2)o1] |
| 97 | 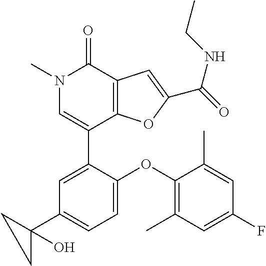 [SMILES: CCNC(=O)c1cc2c(=O)n(C)cc(-c3cc(C4(O)CC4)ccc3Oc3c(C)cc(F)cc3C)c2o1] |
| 98 | 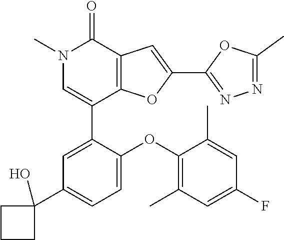 [SMILES: Cc1nnc(-c2cc3c(=O)n(C)cc(-c4cc(C5(O)CCC5)ccc4Oc4c(C)cc(F)cc4C)c3o2)o1] |

In some embodiments, provided herein are compounds described in Table 1, including or a pharmaceutically acceptable salt, hydrate, solvate, isotope, individual isomer, or mixtures of isomers thereof, and uses thereof.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. The structure or name is intended to embrace all possible stereoisomers of a compound depicted, and each unique stereoisomer has a compound number bearing a suffix “a”, “b”, etc. All forms of the compounds are also embraced by the disclosure, such as crystalline or non-crystalline forms of the compounds. Compositions comprising a compound of the disclosure are also intended, such as a composition of substantially pure compound, including a specific stereochemical form thereof, or a composition comprising mixtures of compounds of the disclosure in any ratio, including two or more stereochemical forms, such as in a racemic or non-racemic mixture.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the Formula (I) or variations thereof described herein, where a fraction of one or more atoms are replaced by an isotope of the same element. Exemplary isotopes that can be incorporated into compounds of the disclosure include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O ³⁵S, ¹⁸F ³⁶Cl. Certain isotope labeled compounds (e.g. ³H and ¹⁴C) are useful in compound or substrate tissue distribution study. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present disclosure can generally be prepared by standard methods and techniques known to those skilled in the art or by procedures similar to those described in the accompanying Examples substituting appropriate isotopically-labeled reagents in place of the corresponding non-labeled reagent.

The disclosure also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Methods

The compounds of the disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a pharmaceutically acceptable salt thereof are also contemplated. Solvates contain either stoichiometric or nonstoichiometric amounts of a solvent, and are often formed during the process of crystallization. Hydrates are formed when the solvent is water, or alcoholates are formed when the solvent is alcohol.

Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

Abbreviations used in the descriptions of the schemes and the specific examples have the following meanings: EtOH for ethyl alcohol, B₂Pin₂ for bis(pinacolato)diboron, KOAc for potassium acetate, DMSO for dimethyl sulfoxide, Pd(dppf)Cl₂ for [1,1′-bis(diphenylphosphino)ferrocene]dichloropalladium(II); EtOAc for ethyl acetate; Et₃N for triethylamine; DCM for dichloromethane, DIPEA for N,N-diisopropylethylamine, THF for tetrahydrofuran, T₃P for propylphosphonic anhydride, DMAP for 4-dimethylaminopyridine and HPLC for high-performance liquid chromatography.

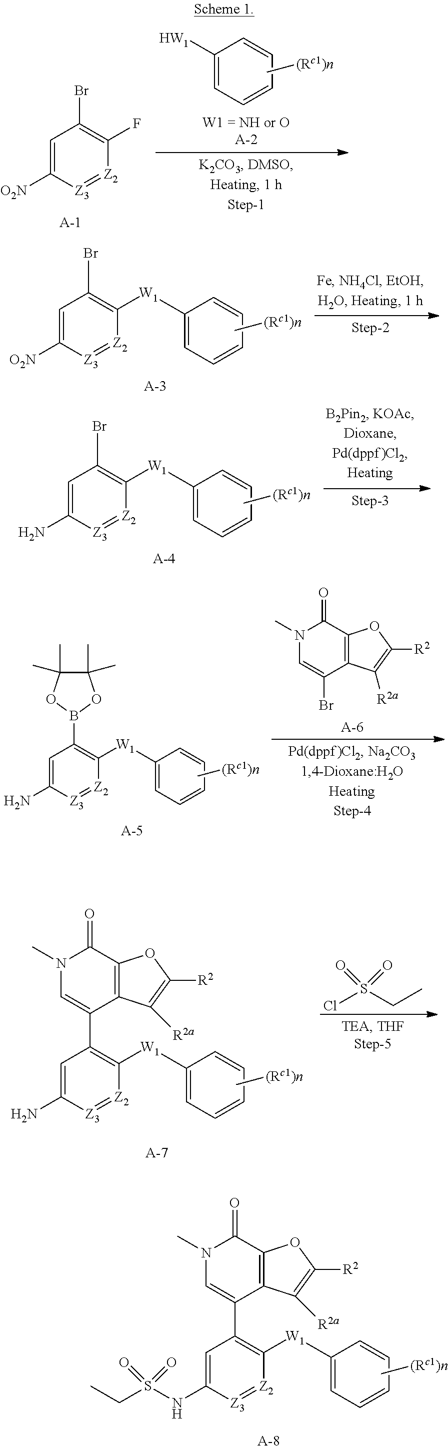

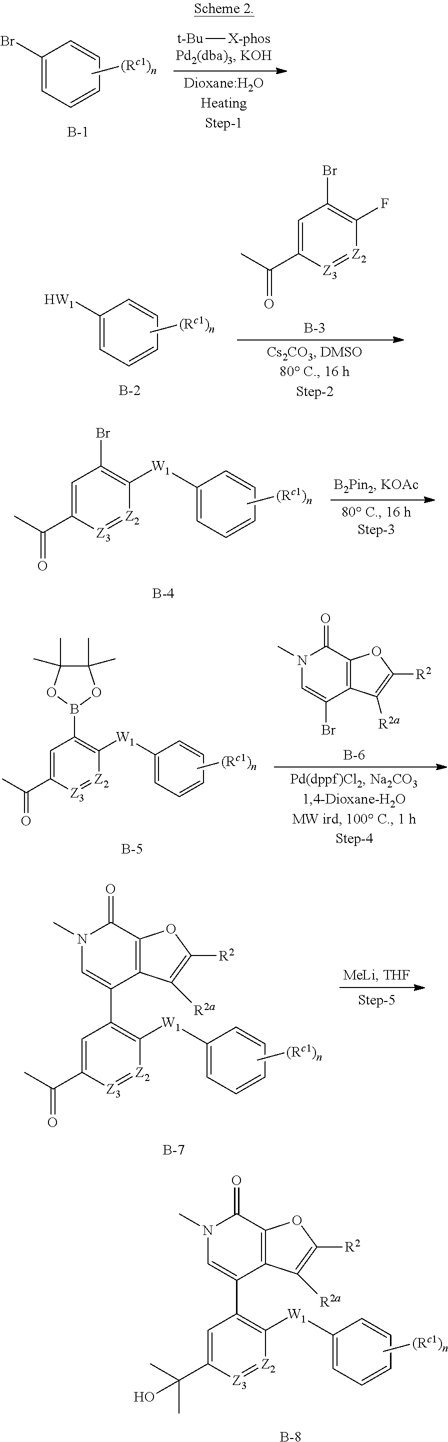

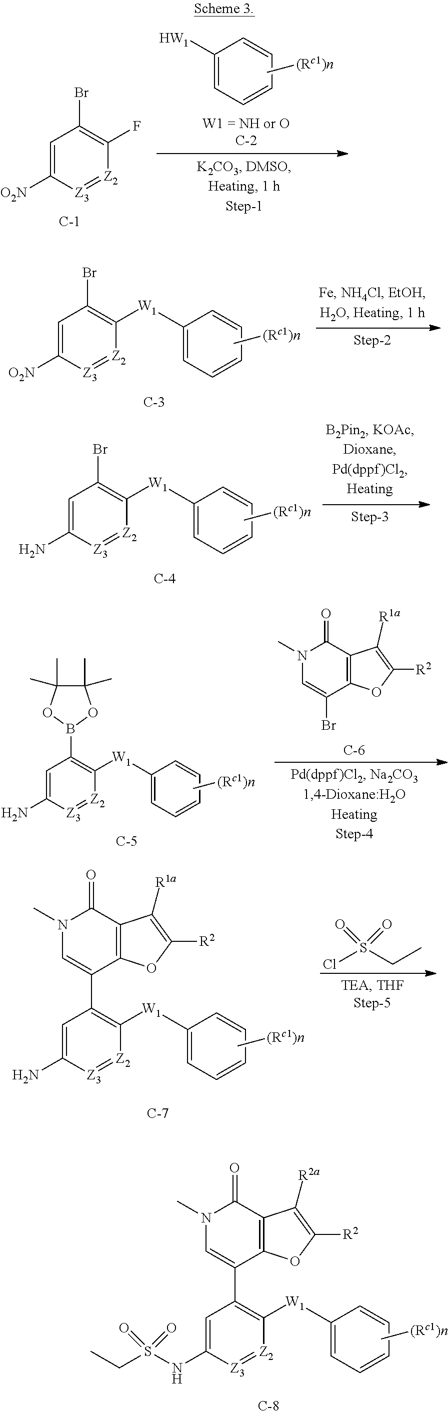

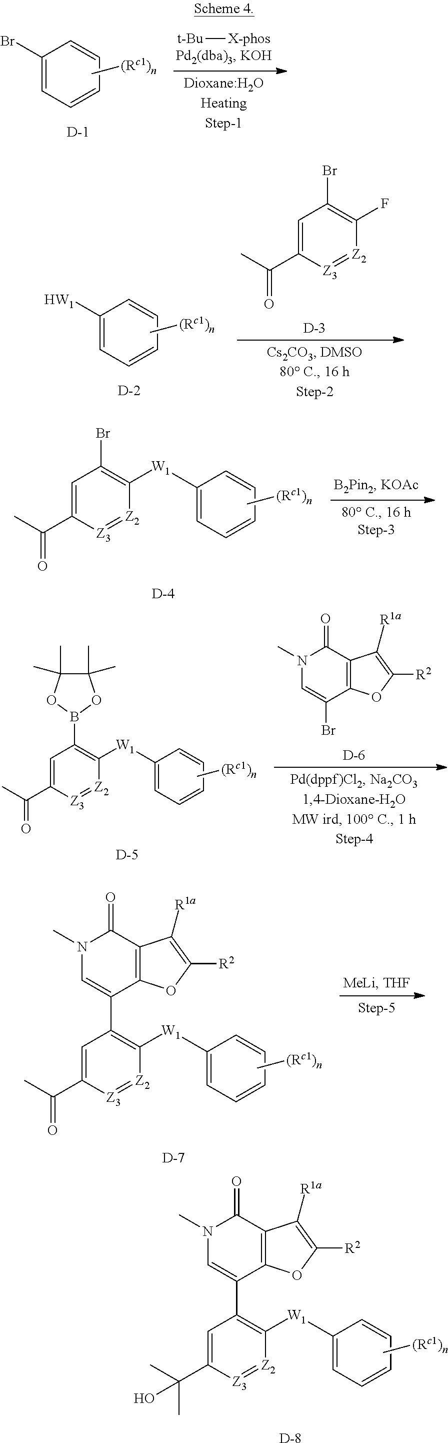

Optimum reaction conditions and reaction times for each individual step may vary depending on the particular reactants employed and substituents present in the reactants used. Unless otherwise specified, solvents, temperatures and other reaction conditions may be readily selected by one of ordinary skill in the art. Specific procedures are provided in the Synthetic Examples section. Reactions may be further processed in the conventional manner, e.g. by eliminating the solvent from the residue and further purified according to methodologies generally known in the art such as, but not limited to, crystallization, distillation, extraction, trituration and chromatography.

Unless otherwise described, the starting materials and reagents are either commercially available or may be prepared by one skilled in the art from commercially available materials using methods described in the chemical literature.

Routine experimentations, including appropriate manipulation of the reaction conditions, reagents and sequence of the synthetic route, protection of any chemical functionality that may not be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety. Synthesis of the compounds of the disclosure may be accomplished by methods analogous to those described in the synthetic schemes described hereinabove and in specific examples.

Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the disclosure is required, it may be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the disclosure is required, it may be obtained by carrying out one of the above procedures using a pure geometric isomer as a starting material, or by resolution of a mixture of the geometric isomers of the compound or intermediates using a standard procedure such as chromatographic separation.

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a pharmaceutically acceptable salt thereof and a pharmaceutically acceptable carrier or excipient. In one aspect, the pharmaceutically acceptable salt is an acid addition salt, such as a salt formed with an inorganic or organic acid.

Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oilin-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc.

Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a pharmaceutically acceptable salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In another embodiment, there are provided methods of making a composition of a compound described herein including formulating a compound of the disclosure with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation. In an embodiment, use of a compound disclosed herein or a pharmaceutically acceptable isomer, racemate, hydrate, solvate, isotope, or salt thereof, for the manufacture of a medicament is provided.

Provided herein is a method of treating a disease in an individual comprising administering an effective amount of a compound disclosed herein (e.g., a compound of Formula (I) or any embodiment, variation or aspect thereof) or a pharmaceutically acceptable salt thereof, to the individual. In some embodiments, provided herein is a method of treating a disease mediated by inhibition of the BET family of proteins in an individual comprising administering an effective amount of a compound disclosed herein, or a pharmaceutically acceptable salt thereof, to the individual. In an embodiment, the present disclosure provides for methods for treating or preventing disorders that are ameliorated by inhibition of BET.

The present compounds or salts thereof are believed to be effective for treating a variety of diseases and disorders. For example, in some embodiments, the present compositions may be used to treat an inflammatory disease, a proliferative disease, such as cancer, or AIDS.

In another aspect, the present disclosure relates to methods of treating cancer in a subject comprising administering a therapeutically effective amount of a compound disclosed herein or a pharmaceutically acceptable salt thereof, to a subject in need thereof. In certain embodiments, the cancer is selected from the group consisting of: acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone sensitive and insensitive prostate cancer, enzalutamide (XTANDI) and abiraterone resistant prostate cancer in the pre- and post-chemo stages, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumors, uterine cancer and Wilms' tumor. In certain embodiments, the methods further comprise administering a therapeutically effective amount of at least one additional therapeutic agent. In certain embodiments, the additional therapeutic agent is an anti-cancer agent. In particular embodiments, the additional therapeutic agents are selected from the group consisting of cytarabine, bortezomib, and 5-azacitidine.

In some embodiments, the cancer is a solid tumor. In some embodiments the cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer, including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

In some embodiments, the cancer in the individual has one or more mutations or amplification or overexpression of the genes encoding BET proteins. In some embodiments, the cancer in the individual has mutation or amplification or overexpression of BRD4. In some embodiments, the cancer in the individual has mutation or amplification or overexpression of c-MYC. In some embodiments, the cancer in the individual has mutation or amplification or overexpression of MYCN. In some embodiments, the cancer in the individual is characterized by Androgen Receptor (AR) expression.

In some embodiments, there is provided a method of treating a cancer in an individual, comprising (a) selecting the individual for treatment based on (i) the mutation or amplification or overexpression of BRD4 or other BET family members, or (ii) presence of mutation or amplification or overexpression of c-MYC in the cancer, and administering an effective amount of a compound disclosed herein or a pharmaceutically acceptable salt thereof, to the individual. In some embodiments, the cancer is sequenced to detect the one or more mutations or amplifications. In some embodiments, the gene is sequenced from the biopsied cancer. In some embodiments, the gene is sequenced by sequencing circulating-tumor DNA (ctDNA) from the individual.

In another aspect, the present disclosure relates to methods of treating a disease or condition in a subject comprising administering a therapeutically effective amount of a compound disclosed herein, or a pharmaceutically acceptable salt thereof, to a subject in need thereof, wherein said disease or condition is selected from the group consisting of: Addison's disease, acute gout, ankylosing spondylitis, asthma, atherosclerosis, Behcet's disease, bullous skin diseases, chronic obstructive pulmonary disease (COPD), Crohn's disease, dermatitis, eczema, giant cell arteritis, glomerulonephritis, hepatitis, hypophysitis, hidradenitis suppurativa, inflammatory bowel disease, Kawasaki disease, liver fibrosis, lupus nephritis, multiple sclerosis, myocarditis, myositis, nephritis, organ transplant rejection, osteoarthritis, pancreatitis, pericarditis, Polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, type I diabetes, ulcerative colitis, uveitis, vitiligo, vasculitis, and Wegener's granulomatosis. In certain embodiments, the methods further comprise administering a therapeutically effective amount of at least one additional therapeutic agent. In certain embodiments, the methods further comprise administering a therapeutically effective amount of at least one additional therapeutic agent.

In another aspect, the present disclosure relates to methods of treating a chronic kidney disease or condition in a subject comprising administering a therapeutically effective amount of a compound disclosed herein, or a pharmaceutically acceptable salt thereof, to a subject in need thereof, wherein said disease or condition is selected from the group consisting of: diabetic nephropathy, hypertensive nephropathy, HIV-associated nephropathy, glomerulonephritis, lupus nephritis, IgA nephropathy, focal segmental glomerulosclerosis, membranous glomerulonephritis, minimal change disease, polycystic kidney disease and tubular interstitial nephritis. In certain embodiments, the methods further comprise administering a therapeutically effective amount of at least one additional therapeutic agent. In certain embodiments, the methods further comprise administering a therapeutically effective amount of at least one additional therapeutic agent.

In another aspect, the present disclosure relates to methods of treating an acute kidney injury or disease or condition in a subject comprising administering a therapeutically effective amount of a compound disclosed herein, or a pharmaceutically acceptable salt thereof, to a subject in need thereof, wherein said acute kidney injury or disease or condition is selected from the group consisting of: ischemia-reperfusion induced, cardiac and major surgery induced, percutaneous coronary intervention induced, radio-contrast agent induced, sepsis induced, pneumonia induced, and drug toxicity induced. In certain embodiments, the methods further comprise administering a therapeutically effective amount of at least one additional therapeutic agent. In certain embodiments, the methods further comprise administering a therapeutically effective amount of at least one additional therapeutic agent.

In another aspect, the present disclosure relates to methods of treating AIDS in a subject comprising administering a therapeutically effective amount of a compound disclosed herein, or a pharmaceutically acceptable salt thereof, to a subject in need thereof. In certain embodiments, the methods further comprise administering a therapeutically effective amount of at least one additional therapeutic agent.

In another aspect, the present disclosure relates to methods of treating obesity, dyslipidemia, hypercholesterolemia, Alzheimer's disease, metabolic syndrome, hepatic steatosis, type II diabetes, insulin resistance, diabetic retinopathy or diabetic neuropathy in a subject comprising administering a therapeutically effective amount of a compound disclosed herein, or a pharmaceutically acceptable salt thereof, to a subject in need thereof. In certain embodiments, the methods further comprise administering a therapeutically effective amount of at least one additional therapeutic agent.

In another aspect, the present disclosure relates to methods of preventing conception by inhibiting spermatogenesis in a subject comprising administering a therapeutically effective amount of a compound disclosed herein, or a pharmaceutically acceptable salt thereof, to a subject in need thereof. In certain embodiments, the methods further comprise administering a therapeutically effective amount of at least one additional therapeutic agent.

### Combination Therapy

As provided herein, the presently disclosed compounds or a salt thereof may be combined with an additional therapeutic agent. In some embodiments, a method of treating a disease in an individual is provided, the method comprising administering an effective amount of a compound disclosed herein, or a pharmaceutically acceptable salt thereof, and an additional therapeutic agent to the individual. In some embodiments, the disease is a proliferative disease such as cancer.

In some embodiments, the additional therapeutic agent is a cancer immunotherapy agent. In some embodiments, the additional therapeutic agent is an immunostimulatory agent. In some embodiments, the additional therapeutic agent targets a checkpoint protein (for example an immune checkpoint inhibitor). In some embodiments, the additional therapeutic agent is effective to stimulate, enhance or improve an immune response against a tumor.

In some embodiments, a method of treating a disease in an individual is provided, the method comprising administering an effective amount of a compound disclosed herein, or a pharmaceutically acceptable salt thereof, in combination with radiation therapy.

In some embodiments, a method of treating a disease in an individual is provided, the method comprising (a) administering an effective amount of a compound disclosed herein, or a pharmaceutically acceptable salt thereof, and (b) administering an effective amount of a chemotherapeutic agent. In some embodiments, a compound disclosed herein, or a pharmaceutically acceptable salt thereof is administered prior to, after, or simultaneously co-administered with the chemotherapeutic agent. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the chemotherapeutic agent.

Examples of chemotherapeutic agents that can be used in combination with a compound disclosed herein, or a pharmaceutically acceptable salt thereof include a DNA alkylating agent (such as cyclophosphamide, mechlorethamine, chlorambucil, melphalan, dacarbazine, or nitrosoureas), a topoisomerase inhibitor (such as a Topoisomerase I inhibitor (e.g., irinotecan or topotecan) or a Topoisomerase II inhibitor (e.g., etoposide or teniposide), an anthracycline (such as daunorubicin, doxorubicin, epirubicin, idarubicin, mitoxantrone, or valrubicin), a histone deacetylase inhibitor (such as vorinostat or romidepsin), another bromodomain inhibitor, other epigenetic inhibitors, a taxane (such as paclitaxel or docetaxel), a kinase inhibitor (such as bortezomib, erlotinib, gefitinib, imatinib, vemurafenib, vismodegib, ibrutinib), a mTOR inhibitor, a DNA Damage Repair (DDR) pathway inhibitor, such as a PARP inhibitor, ATM inhibitor, ATR inhibitor, a Weel inhibitor, a proteasome inhibitor (such as bortezomib), an anti-angiogenic inhibitor, endocrine therapy, anti-estrogen therapy, anti-androgen therapy, glucocorticoid receptor inhibitor, a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine), or a platinum-based chemotherapeutic agent (such as cisplatin, carboplatin, or oxaliplatin), pemetrexed, or a combination thereof.

In some embodiments, a method of treating a disease in an individual is provided, the method comprising (a) administering an effective amount of a compound disclosed herein, or a pharmaceutically acceptable salt thereof, and (b) administering an effective amount of a DNA damaging agent. In some embodiments, a compound disclosed herein, or a pharmaceutically acceptable salt thereof is administered prior to, after, or simultaneously co-administered with the DNA damaging agent. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the DNA damaging agent.

In some embodiments, a method of treating a disease in an individual is provided, the method comprising (a) administering an effective amount of a compound disclosed herein, or a pharmaceutically acceptable salt thereof, and (b) administering an effective amount of a DNA alkylating agent (such as cyclophosphamide, mechlorethamine, chlorambucil, melphalan, dacarbazine, or nitrosoureas). In some embodiments, a compound disclosed herein, or a pharmaceutically acceptable salt thereof is administered prior to, after, or simultaneously co-administered with the DNA alkylating agent. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the DNA alkylating agent.

In some embodiments, a method of treating a disease in an individual is provided, the method comprising (a) administering an effective amount of a compound disclosed herein, or a pharmaceutically acceptable salt thereof, and (b) administering an effective amount of a topoisomerase inhibitor (such as a Topoisomerase I inhibitor (e.g., irinotecan or topotecan) or a Topoisomerase II inhibitor (e.g., etoposide or teniposide)). In some embodiments, a compound disclosed herein, or a pharmaceutically acceptable salt thereof is administered prior to, after, or simultaneously co-administered with the topoisomerase inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the topoisomerase inhibitor.

In some embodiments, a method of treating a disease in an individual is provided, the method comprising (a) administering an effective amount of a compound disclosed herein, or a pharmaceutically acceptable salt thereof, and (b) administering an effective amount of an anthracycline (such as daunorubicin, doxorubicin, epirubicin, idarubicin, mitoxantrone, or valrubicin). In some embodiments, a compound disclosed herein, or a pharmaceutically acceptable salt thereof is administered prior to, after, or simultaneously co-administered with the anthracycline. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the anthracycline.

In some embodiments, a method of treating a disease in an individual is provided, the method comprising (a) administering an effective amount of a compound disclosed herein, or a pharmaceutically acceptable salt thereof, and (b) administering an effective amount of a histone deacetylase inhibitor (such as vorinostat or romidepsin). In some embodiments, a compound disclosed herein, or a pharmaceutically acceptable salt thereof is administered prior to, after, or simultaneously co-administered with the histone deacetylase inhibitor. In some embodiments, A compound disclosed herein, or a pharmaceutically acceptable salt thereof, is administered 1 or more hours (such as 2 or more hours, 4 or more hours, 8 or more hours, 12 or more hours, 24 or more hours, or 48 or more hours) prior to or after the histone deacetylase inhibitor.

In some embodiments, a method of treating a disease in an individual is provided, the method comprising (a) administering an effective amount of a compound disclosed herein, or a pharmaceutically acceptable salt thereof, and (b) administering an effective amount of a taxane (such as paclitaxel or docetaxel). In some embodiments, a compound disclosed herein, or a pharmaceutically acceptable salt thereof is administered prior to, after, or simultaneously co-administered with the taxane. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the taxane.

In some embodiments, a method of treating a disease in an individual is provided, the method comprising (a) administering an effective amount of a compound disclosed herein, or a pharmaceutically acceptable salt thereof, and (b) administering an effective amount of a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine). In some embodiments, a compound disclosed herein, or a pharmaceutically acceptable salt thereof is administered prior to, after, or simultaneously co-administered with the nucleotide analog or precursor analog. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the nucleotide analog or precursor analog.

In some embodiments, a method of treating a disease in an individual is provided, the method comprising (a) administering an effective amount of a compound disclosed herein, or a pharmaceutically acceptable salt thereof, and (b) administering an effective amount of a platinum-based chemotherapeutic agent (such as cisplatin, carboplatin, or oxaliplatin). In some embodiments, a compound disclosed herein, or a pharmaceutically acceptable salt thereof is administered prior to, after, or simultaneously co-administered with the platinum-based chemotherapeutic agent. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the platinum-based chemotherapeutic agent.

In some embodiments, a method of treating a disease in an individual is provided, the method comprising (a) administering an effective amount of a compound disclosed herein, or a pharmaceutically acceptable salt thereof, and (b) administering an effective amount of pemetrexed. In some embodiments, a compound disclosed herein, or a pharmaceutically acceptable salt thereof is administered prior to, after, or simultaneously co-administered with the pemetrexed. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the pemetrexed.

In some embodiments, a method of treating a disease in an individual is provided, the method comprising (a) administering an effective amount of a compound disclosed herein, or a pharmaceutically acceptable salt thereof, and (b) administering an effective amount of a kinase inhibitor (such as bortezomib, erlotinib, gefitinib, imatinib, vemurafenib, vismodegib, or ibrutinib). In some embodiments, a compound disclosed herein, or a pharmaceutically acceptable salt thereof is administered prior to, after, or simultaneously co-administered with the kinase inhibitor. In some embodiments, Formula I or a pharmaceutically acceptable salt thereof is administered 1 or more hours (such as 2 or more hours, 4 or more hours, 8 or more hours, 12 or more hours, 24 or more hours, or 48 or more hours) prior to or after the kinase inhibitor.

In some embodiments, a method of treating a disease in an individual is provided, the method comprising (a) administering an effective amount of a compound disclosed herein, or a pharmaceutically acceptable salt thereof, and (b) administering an effective amount of a mTOR inhibitor (such as everolimus). In some embodiments, a compound disclosed herein, or a pharmaceutically acceptable salt thereof is administered prior to, after, or simultaneously co-administered with the mTOR inhibitor.

In some embodiments, a method of treating a disease in an individual is provided, the method comprising (a) administering an effective amount of a compound disclosed herein, or a pharmaceutically acceptable salt thereof, and (b) administering an effective amount of a PI3K or Akt inhibitor. In some embodiments, a compound disclosed herein, or a pharmaceutically acceptable salt thereof is administered prior to, after, or simultaneously co-administered with the PI3K or Akt inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the PI3K or Akt inhibitor.

In some embodiments, a method of treating a disease in an individual is provided, the method comprising (a) administering an effective amount of a compound disclosed herein, or a pharmaceutically acceptable salt thereof, and (b) administering an effective amount of a Bruton's tyrosine kinase (BTK) inhibitor. In some embodiments, a compound disclosed herein, or a pharmaceutically acceptable salt thereof is administered prior to, after, or simultaneously co-administered with the BTK inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the BTK inhibitor.

In some embodiments, a method of treating a disease in an individual is provided, the method comprising (a) administering an effective amount of a compound disclosed herein, or a pharmaceutically acceptable salt thereof, and (b) administering an effective amount of a Cyclin-dependent kinase (CDK) inhibitor, such as inhibitor of CDK1, CDK2, CDK4, CDK5, CDK6, CDK7, or CDK9, or any combination thereof. In some embodiments, a compound disclosed herein, or a pharmaceutically acceptable salt thereof is administered prior to, after, or simultaneously co-administered with the CDK inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the CDK inhibitor.

In some embodiments, a method of treating a disease in an individual is provided, the method comprising (a) administering an effective amount of a compound disclosed herein, or a pharmaceutically acceptable salt thereof, and (b) administering an effective amount of a DNA damage repair (DDR) pathway inhibitor. In some embodiments, a compound disclosed herein, or a pharmaceutically acceptable salt thereof is administered prior to, after, or simultaneously co-administered with the DDR pathway inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the DDR pathway inhibitor. Examples of inhibitors of the DDR pathway include poly(ADP-ribose) polymerase (PARP) inhibitors (such as olaparib, rucaparib, niraparib, or talazoparib), ataxia telangiectasia mutated (ATM) protein inhibitors, ataxia telangiectasia and Rad3-related (ATR) protein inhibitors, checkpoint kinase 1 (Chk1) inhibitors, or combinations thereof.

In some embodiments, a method of treating a disease in an individual is provided, the method comprising (a) administering an effective amount of a compound disclosed herein, or a pharmaceutically acceptable salt thereof, and (b) administering an effective amount of a PARP inhibitor (such as olaparib, rucaparib, niraparib, or talazoparib). In some embodiments, a compound disclosed herein, or a pharmaceutically acceptable salt thereof, is administered prior to, after, or simultaneously co-administered with the PARP inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the PARP inhibitor.

In some embodiments, a method of treating a disease in an individual is provided, the method comprising (a) administering an effective amount of a compound disclosed herein, or a pharmaceutically acceptable salt thereof, and (b) administering an effective amount of an ATM protein inhibitor. In some embodiments, a compound disclosed herein, or a pharmaceutically acceptable salt thereof is administered prior to, after, or simultaneously co-administered with the ATM protein inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the ATM protein inhibitor.

In some embodiments, a method of treating a disease in an individual is provided, the method comprising (a) administering an effective amount of a compound disclosed herein, or a pharmaceutically acceptable salt thereof, and (b) administering an effective amount of an ATR protein inhibitor. In some embodiments, a compound disclosed herein, or a pharmaceutically acceptable salt thereof is administered prior to, after, or simultaneously co-administered with the ATR protein inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the ATR protein inhibitor.

In some embodiments, a method of treating a disease in an individual is provided, the method comprising (a) administering an effective amount of a compound disclosed herein, or a pharmaceutically acceptable salt thereof, and (b) administering an effective amount of a Chk1 inhibitor. In some embodiments, a compound disclosed herein, or a pharmaceutically acceptable salt thereof is administered prior to, after, or simultaneously co-administered with the Chk1 inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the Chk1 inhibitor.

In some embodiments, a method of treating a disease in an individual is provided, the method comprising (a) administering an effective amount of a compound disclosed herein, or a pharmaceutically acceptable salt thereof, and (b) administering an effective amount of a Weel inhibitor. In some embodiments, a compound disclosed herein, or a pharmaceutically acceptable salt thereof is administered prior to, after, or simultaneously co-administered with the Weel inhibitor. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the Wee1 inhibitor.

In some embodiments, a method of treating a disease in an individual is provided, the method comprising (a) administering an effective amount of a compound disclosed herein, or a pharmaceutically acceptable salt thereof, and (b) administering an effective amount of an endocrine therapy agent. In some embodiments, the endocrine therapy is antiestrogen therapy. In some embodiments, the endocrine therapy is a selective estrogen receptor degrader (SERD, such as fulvestrant). In some embodiments, the endocrine therapy is an aromatase inhibitor (such as letrozole). In some embodiments, the endocrine therapy is an anti-androgen therapy (such as enzalutamide or apalutamide). In some embodiments, the endocrine therapy is a CYP17 inhibitor (such as abiraterone). In some embodiments, a compound disclosed herein, or a pharmaceutically acceptable salt thereof is administered prior to, after, or simultaneously co-administered with the endocrine therapy agent. In some embodiments, a compound disclosed herein, or a pharmaceutically acceptable salt thereof is administered 1 or more hours (such as 2 or more hours, 4 or more hours, 8 or more hours, 12 or more hours, 24 or more hours, or 48 or more hours) prior to or after the endocrine therapy agent.

In another aspect, provided herein is a combination therapy in which a compound of a compound disclosed herein, or a pharmaceutically acceptable salt thereof is coadministered (which may be separately or simultaneously) with one or more additional agents that are effective in stimulating immune responses to thereby further enhance, stimulate or upregulate immune responses in a subject. For example, provided is a method for stimulating an immune response in a subject comprising administering to the subject a compound disclosed herein, or a pharmaceutically acceptable salt thereof, and one or more immunostimulatory antibodies, such as an anti-PD-1 antibody, an anti-PD-L1 antibody and/or an anti-CTLA-4 antibody, such that an immune response is stimulated in the subject, for example to inhibit tumor growth. In one embodiment, the subject is administered a compound disclosed herein, or a pharmaceutically acceptable salt thereof, and an anti-PD-1 antibody. In another embodiment, the subject is administered a compound disclosed herein, or a pharmaceutically acceptable salt thereof, and an anti-PD-L1 antibody. In yet another embodiment, the subject is administered a compound disclosed herein, or a pharmaceutically acceptable salt thereof, and an anti-CTLA-4 antibody. In another embodiment, the immunostimulatory antibody (e.g., anti-PD-1, anti-PD-L1 and/or anti-CTLA-4 antibody) is a human antibody. Alternatively, the immunostimulatory antibody can be, for example, a chimeric or humanized antibody (e.g., prepared from a mouse anti-PD-1, anti-PD-L1 and/or anti-CTLA-4 antibody).

In one embodiment, the present disclosure provides a method for treating a proliferative disease (e.g., cancer), comprising administering a compound disclosed herein, or a pharmaceutically acceptable salt thereof, and an anti-PD-1 antibody or to a subject. In further embodiments, a compound disclosed herein, or a pharmaceutically acceptable salt thereof, is administered at a subtherapeutic dose, the anti-PD-1 antibody is administered at a subtherapeutic dose, or both are administered at a subtherapeutic dose. In another embodiment, the present disclosure provides a method for altering an adverse event associated with treatment of a hyperproliferative disease with an immunostimulatory agent, comprising administering a compound disclosed herein, or a pharmaceutically acceptable salt thereof, and a subtherapeutic dose of anti-PD-1 antibody to a subject. In certain embodiments, the subject is human. In certain embodiments, the anti-PD-1 antibody is a human sequence monoclonal antibody.

In one embodiment, the present disclosure provides a method for treating a hyperproliferative disease (e.g., cancer), comprising administering a compound disclosed herein, or a pharmaceutically acceptable salt thereof, and an anti-PD-L1 antibody to a subject. In further embodiments, a compound disclosed herein, or a pharmaceutically acceptable salt thereof, is administered at a subtherapeutic dose, the anti-PD-L1 antibody is administered at a subtherapeutic dose, or both are administered at a subtherapeutic dose. In another embodiment, the present disclosure provides a method for altering an adverse event associated with treatment of a hyperproliferative disease with an immunostimulatory agent, comprising administering a compound disclosed herein, or a pharmaceutically acceptable salt thereof, and a subtherapeutic dose of anti-PD-L1 antibody to a subject. In certain embodiments, the subject is human. In certain embodiments, the anti-PD-L1 antibody is a human sequence monoclonal antibody.

In certain embodiments, the combination of therapeutic agents discussed herein can be administered concurrently as a single composition in a pharmaceutically acceptable carrier, or concurrently as separate compositions each in a pharmaceutically acceptable carrier. In another embodiment, the combination of therapeutic agents can be administered sequentially. For example, an anti-CTLA-4 antibody and a compound disclosed herein, or a pharmaceutically acceptable salt thereof, can be administered sequentially, such as anti-CTLA-4 antibody being administered first and a compound disclosed herein, or a pharmaceutically acceptable salt thereof, second, or a compound disclosed herein, or a pharmaceutically acceptable salt thereof, being administered first and anti-CTLA-4 antibody second. Additionally or alternatively, an anti-PD-1 antibody and a compound disclosed herein, or a pharmaceutically acceptable salt thereof, can be administered sequentially, such as anti-PD-1 antibody being administered first and a compound disclosed herein, or a pharmaceutically acceptable salt thereof, second, or a compound disclosed herein, or a pharmaceutically acceptable salt thereof, being administered first and anti-PD-1 antibody second. Additionally or alternatively, an anti-PD-L1 antibody and a compound disclosed herein, or a pharmaceutically acceptable salt thereof, can be administered sequentially, such as anti-PD-L1 antibody being administered first a compound disclosed herein, or a pharmaceutically acceptable salt thereof, second, or a compound disclosed herein, or a pharmaceutically acceptable salt thereof, being administered first and anti-PD-L1 antibody second.

Furthermore, if more than one dose of the combination therapy is administered sequentially, the order of the sequential administration can be reversed or kept in the same order at each time point of administration, sequential administrations can be combined with concurrent administrations, or any combination thereof.

Optionally, the combination of a compound disclosed herein, or a pharmaceutically acceptable salt thereof, can be further combined with an immunogenic agent, such as cancerous cells, purified tumor antigens (including recombinant proteins, peptides, and carbohydrate molecules), cells, and cells transfected with genes encoding immune stimulating cytokines.

A compound disclosed herein, or a pharmaceutically acceptable salt thereof, can also be further combined with standard cancer treatments. For example, a compound disclosed herein, or a pharmaceutically acceptable salt thereof, can be effectively combined with chemotherapeutic regimens. In these instances, it is possible to reduce the dose of other chemotherapeutic reagent administered with the combination of the instant disclosure. Other combination therapies with a compound disclosed herein, or a pharmaceutically acceptable salt thereof, include radiation, surgery, or hormone deprivation. Angiogenesis inhibitors can also be combined with a compound disclosed herein, or a pharmaceutically acceptable salt thereof. Inhibition of angiogenesis leads to tumor cell death, which can be a source of tumor antigen fed into host antigen presentation pathways.

In another example, a compound disclosed herein, or a pharmaceutically acceptable salt thereof, can be used in conjunction with anti-neoplastic antibodies. By way of example and not wishing to be bound by theory, treatment with an anti-cancer antibody or an anti-cancer antibody conjugated to a toxin can lead to cancer cell death (e.g., tumor cells) which would potentiate an immune response mediated by CTLA-4, PD-1, PD-L1 or a compound disclosed herein, or a pharmaceutically acceptable salt thereof. In an exemplary embodiment, a treatment of a hyperproliferative disease (e.g., a cancer tumor) can include an anti-cancer antibody in combination with a compound disclosed herein, or a pharmaceutically acceptable salt thereof, and anti-CTLA-4 and/or anti-PD-1 and/or anti-PD-L1 antibodies, concurrently or sequentially or any combination thereof, which can potentiate anti-tumor immune responses by the host. Other antibodies that can be used to activate host immune responsiveness can be further used in combination with a compound disclosed herein, or a pharmaceutically acceptable salt thereof.

In yet further embodiments, a compound disclosed herein, or a pharmaceutically acceptable salt thereof, is administered in combination with another BET inhibitor.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the disclosure may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as “metronomic therapy,” or as part of a maintenance therapy using compound alone or in combination with one or more additional drugs. Metronomic therapy or maintenance therapy can comprise administration of a compound provided herein in cycles. Metronomic therapy or maintenance therapy can comprise intra-humoral administration of a compound provided herein.

In one aspect, the disclosure provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The disclosure also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form.

Also provided are articles of manufacture comprising a compound of the disclosure or a salt thereof, composition, and unit dosages described herein in suitable packaging for use in the methods described herein. Suitable packaging is known in the art and includes, for example, vials, vessels, ampules, bottles, jars, flexible packaging and the like. An article of manufacture may further be sterilized and/or sealed kits.

The present disclosure further provides kits for carrying out the methods of the disclosure, which comprises one or more compounds described herein or a composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or subunit doses. For example, kits may be provided that contain sufficient dosages of a compound as disclosed herein and/or a second pharmaceutically active compound useful for a disease detailed herein (e.g., hypertension) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

The disclosure can be further understood by reference to the following examples, which are provided by way of illustration and are not meant to be limiting

### SYNTHETIC EXAMPLES

### Example S-1: 1-(4-(4-fluoro-2,6-dimethylphenoxy)-3-(4,4,5,5-tetramethyl-1,3,2-dioxaborolan-2-yl)phenyl)ethan-1-one

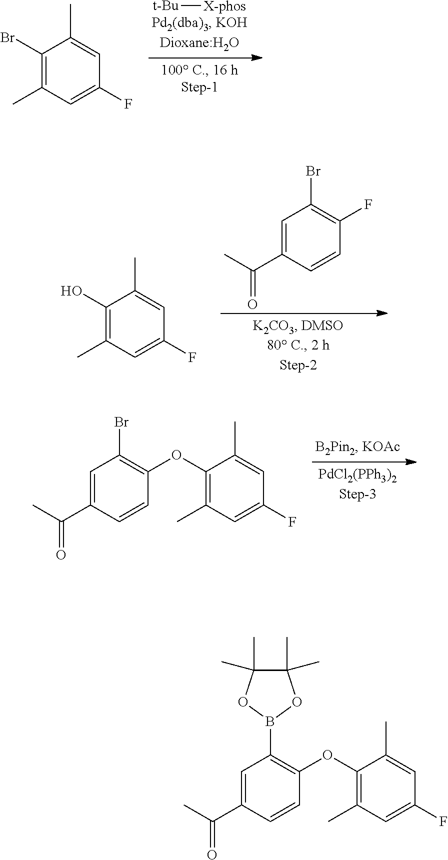

Step 1. Synthesis of 4-fluoro-2,6-dimethylphenol. A solution of 2-bromo-5-fluoro-1,3-dimethylbenzene (5.0 g, 24.7 mmol, 1 eq) in 1,4-dioxane:water (50 mL, 1:1) was added KOH (4.15 g, 74.2 mmol, 3 eq) and the mixture was degassed under nitrogen for 15 min. In another set-up, t-Bu-X-phos (839 mg, 7.98 mmol 0.08 eq) and Pd₂(dba)₃ (452 mg, 0.49 mmol, 0.08 eq) in 1,4-dioxane:water (20 mL, 1:1) was degassed under nitrogen for 15 min. The contents of the first degassed mixture was transferred into the degassed solution of the second and the mixture was heated at 100° C. and monitored by TLC and LCMS. The reaction was complete after 16 h and the mixture was acidified with 6N—HCl (pH ˜2-3) and extracted with EtOAc (700 mL). The organic layer was washed with water (300 mL), brine (200 mL), dried over anhydrous Na₂SO₄, filtered and concentrated under reduced pressure to obtain a crude residue which was purified by CombiFlash chromatography to afford the title compound. LCMS: 141 [M+H]⁺.

Step 2. Synthesis of 1-(3-bromo-4-(4-fluoro-2,6-dimethylphenoxy)phenyl)-ethanone. To a solution of 4-fluoro-2,6-dimethylphenol (0.50 g, 3.57 mmol) in DMSO (20 mL) was added K₂CO₃ (0.98 g, 7.15 mmol, 2 eq) at RT and the mixture was stirred for 15 min. 1-(3-bromo-4-fluorophenyl)ethanone (0.93 g, 4.28 mmol, 1.2 eq) was then added to the mixture and the resultant mixture was heated 80° C. for 2 h. The reaction was complete after 2 h and to the mixture was added water (200 mL) to obtain a precipitate which was filtered over Büchner funnel; dried under vacuum to afford the title compound. LCMS: 337 [M+H]⁺, 339 [M+2+H]⁺.

Step 3. Synthesis of 1-(4-(4-fluoro-2,6-dimethylphenoxy)-3-(4,4,5,5-tetramethyl-1,3,2-dioxaborolan-2-yl)phenyl)ethenone. To a solution of 1-(3-bromo-4-(4-fluoro-2,6-dimethylphenoxy)phenyl)ethanone (0.55 g, 1.63 mmol, 1 eq) in 1,4-dioxane (5 mL), was added B₂Pin₂ (0.50 g, 1.96 mmol, 1.2 eq), KOAc (0.48 g, 4.89 mmol, 3 eq), and Pd(dppf)Cl₂ (0.12 g, 0.16 mmol, 0.1 eq). The reaction mixture was degassed and purged with N2. Then the mixture was stirred at overnight at 80° C. TLC analysis indicated the reaction was complete. The mixture was concentrated under reduced pressure. The residue was purified by column chromatography on silica gel to afford the title compound.

### Example S-2: Synthesis of N-ethyl-7-(2-(4-fluoro-2,6-dimethylphenoxy)-5-(2-hydroxypropan-2-yl)phenyl)-5-methyl-4-oxo-4,5-dihydrofuro[3,2-c]pyridine-2-carboxamide Compound 1

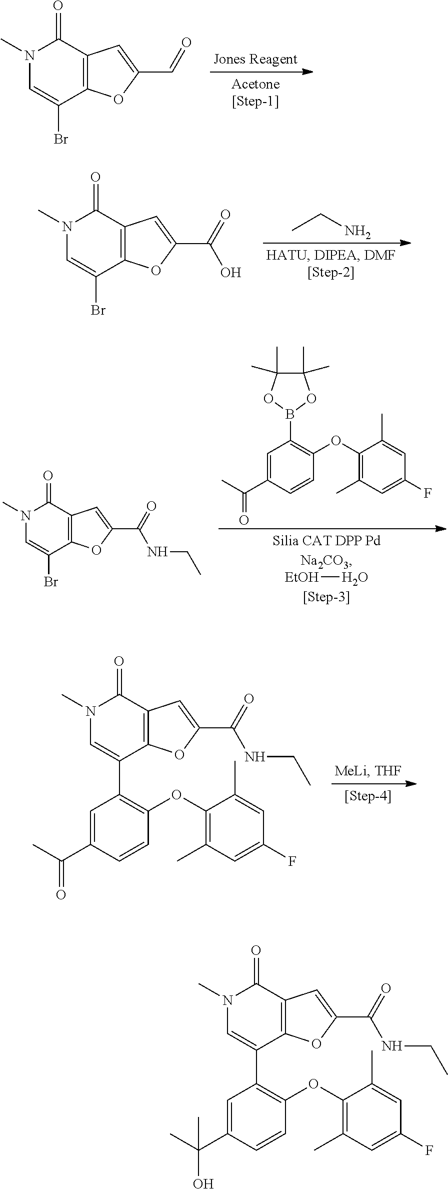

Step 1. Synthesis of 7-bromo-5-methyl-4-oxo-4,5-dihydrofuro[3,2-c]pyridine-2-carboxylic acid: A solution of 7-bromo-5-methyl-4-oxo-4,5-dihydrofuro[3,2-c]pyridine-2-carbaldehyde (1.0 g, 3.9 mmol, 1 eq) in acetone (50 mL) was added Jones reagent (2 mL) at 0° C. dropwise over a period of 20 min. The resultant mixture was stirred at 0° C. for 5 min. The reaction was monitored by TLC. After completion, water (200 mL) was added and the resulting precipitate was filtered over BUchner funnel. The solid obtained was washed with water (50 mL×2) and n-pentane (25 mL×2), dried under vacuum to afford the title compound which was taken to next step without further purification. LCMS: 272 [M+H]⁺, 274 [M+2+H]⁺.

Step 2. Synthesis of 7-bromo-N-ethyl-5-methyl-4-oxo-4,5-dihydrofuro[3,2-c]pyridine-2-carboxamide: To a stirred solution of 7-bromo-5-methyl-4-oxo-4,5-dihydrofuro[3,2-c] pyridine-2-carboxylic acid (0.2 g, 0.73 mmol, 1 eq) in DMF (6 mL) were successively added HATU (0.42 g, 1.47 mmol, 1.5 eq) and DIPEA (0.76 mL, 4.4 mmol, 6 eq) at 0° C. and the mixture was stirred at same temperature 20 min. Ethanamine (0.18 g, 2.2 mmol, 3.0 eq) was then added to the mixture and the resultant mixture was stirred at RT for 16 h. The reaction was complete after 16 h. The mixture was diluted with water (30 mL) and extracted with EtOAc (30 mL×2). The combined organic layers were washed with water (30 mL), brine (25 mL), dried over anhydrous Na₂SO₄, filtered and concentrated under reduced pressure to obtain a crude which was purified by CombiFlash chromatography to afford the title compound. LCMS: 299 [M+H]⁺, 301 [M+2+H]⁺.

Step 3. Synthesis of 7-(5-acetyl-2-(4-fluoro-2,6-dimethylphenoxy)phenyl)-N-ethyl-5-methyl-4-oxo-4,5-dihydrofuro[3,2-c]pyridine-2-carboxamide: To a stirred solution of 7-bromo-N-ethyl-5-methyl-4-oxo-4,5-dihydrofuro[3,2-c]pyridine-2-carboxamide (0.15 g, 0.50 mmol, 1 eq) in ethanol (9 mL) were added 1-(4-(4-fluoro-2,6-dimethylphenoxy)-3-(4,4,5,5-tetra methyl-1,3,2-dioxaborolan-2-yl)phenyl)ethanone (0.23 g, 0.60 mmol, 1.2 eq) and Na₂CO₃ (0.11 g, 1.0 mmol, 2 eq) dissolved in water (1 mL). Silia CAT DPP-Pd (0.30 mmol/g loading; 0.08 g, 0.025 mmol, 0.05 eq) was then added to the mixture and the resultant mixture was then heated at 100° C. for 2 h. The reaction was complete after 2 h and the mixture was filtered through the celite bed and washed with 5% MeOH in DCM (30 mL). The filtrate obtained was concentrated under reduced pressure to obtain a crude residue. The residue obtained was stirred in MeOH (5 mL) for 20 min, collected on a Buchner funnel and dried under vacuum to obtain a solid compound. Then solid obtained was further triturated with diethyl ether (5 mL) and dried to afford title compound. LCMS: 477 [M+H]⁺.

Step 4. Synthesis of N-ethyl-7-(2-(4-fluoro-2,6-dimethylphenoxy)-5-(2-hydroxypropan-2-yl)phenyl)-5-methyl-4-oxo-4,5-dihydrofuro[3,2-c]pyridine-2-carboxamide: To a stirred solution of 7-(5-acetyl-2-(4-fluoro-2,6-dimethylphenoxy)phenyl)-N-ethyl-5-methyl-4-oxo-4,5-dihydrofuro[3,2-c]pyridine-2-carboxamide (0.09 g, 0.19 mmol, 1 eq) in anhydrous THF (5 mL) was added methyl lithium (1.6 M in Et₂O; 0.6 mL, 0.94 mmol, 6 eq) at 0° C. dropwise and the mixture was stirred at same temperature for 10 min. The progress of the reaction was monitored by TLC. After 10 min and the mixture was slowly quenched with a saturated NH₄Cl solution (4 mL). The aqueous layer was then extracted with EtOAc (50 mL×2). The combined organic layers were washed with water (50 mL), brine (30 mL), dried over anhydrous Na₂SO₄, filtered and concentrated under reduced pressure to obtain a crude which was purified by reversed phase HPLC to afford the title compound. LCMS: 493 [M+H]⁺; ¹H NMR (400 MHz, CD₃OD): δ 7.83 (s, 1H), 7.68-7.58 (m, 2H), 7.39 (dd, J=2.6, 8.8 Hz, 1H), 6.84 (d, J=8.8 Hz, 2H), 6.38 (d, J=8.8 Hz, 1H), 3.73 (s, 3H), 3.42-3.33 (m, 2H), 2.07 (s, 6H), 1.55 (s, 6H), 1.18 (t, J=7.2 Hz, 3H).

### Example S-3: Synthesis of 4-(2,4-difluorophenoxy)-3-(4,4,5,5-tetramethyl-1,3,2-dioxaborolan-2-yl)aniline

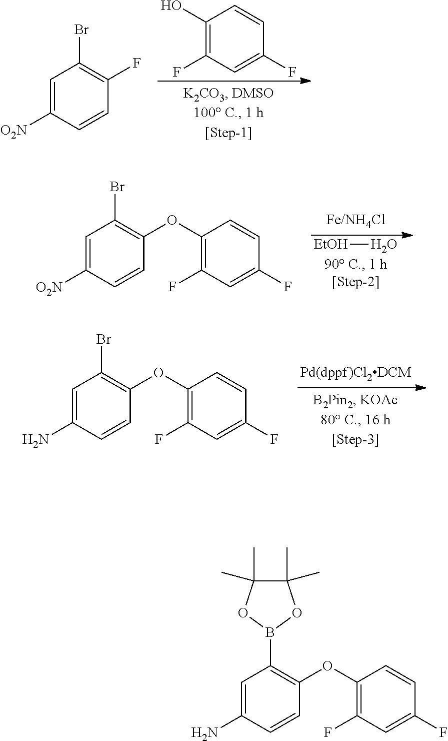

Step 1: Synthesis of 2-bromo-1-(2,4-difluorophenoxy)-4-nitrobenzene: To a stirred solution of 2,4-difluorophenol (3.0 g, 23 mmol, 1 eq) in DMSO (20 mL) was added K₂CO₃ (3.0 g, 46 mmol, 2 eq) at RT followed by the addition of 2-bromo-1-fluoro-4-nitrobenzene (5.6 g, 25.3 mmol, 1.1 eq) and the mixture was heated at 100° C. for 1 h. The reaction was complete after 2 h and to the mixture was added ice-cold water (100 mL) to obtain a precipitate which was filtered over Büchner funnel and dried under vacuum to afford the title compound.

Step 2: Synthesis of 3-bromo-4-(2,4-difluorophenoxy)aniline: To a solution of 2-bromo-1-(2,4-difluorophenoxy)-4-nitrobenzene (6 g, 18.2 mmol, 1 eq) in ethanol (50 mL), a solution of NH₄Cl (7.8 g, 145.4 mmol, 8 eq) in water (50 mL) was added followed by addition of iron powder (5.1 g, 91 mmol, 5 eq). The resultant mixture was heated at 90° C. for 1 h. The reaction was monitored by TLC. Upon completion, the mixture was filtered through a pad of Celite and concentrated under reduced pressure. The residue obtained was diluted with water (200 mL) and extracted with EtOAc (300 mL×2). The combined organic layers were washed with brine (100 mL), dried over anhydrous Na₂SO₄, filtered and concentrated under reduced pressure to obtain a crude residue which was purified by CombiFlash chromatography to afford the title compound.

Step 3: Synthesis of 4-(2,4-difluorophenoxy)-3-(4,4,5,5-tetramethyl-1,3,2-dioxaborolan-2-yl)aniline: To a solution of 3-bromo-4-(2,4-difluorophenoxy)aniline (4.0 g, 13.4 mmol, 1 eq) in 1,4-dioxane (40 mL) was added B₂Pin₂ (5.1 g, 20.1 mmol, 1.5 eq), KOAc (8.5 g, 33.6 mmol, 2.5 eq) and the mixture was degassed under N2 for 20 min. Pd(dppf)Cl₂.DCM (1.1 g, 1.34 mmol, 0.1 eq) was then added to the mixture and the resultant mixture was heated at 80° C. for 16 h. The reaction was monitored by TLC. Upon completion, the mixture was filtered through a pad of Celite and concentrated under reduced pressure. The residue obtained was diluted with water (200 mL) and extracted with EtOAc (250 mL×2). The combined organic layers were washed with water (200 mL), brine (100 mL), dried over anhydrous Na₂SO₄, filtered and concentrated under reduced pressure to obtain a crude residue which was purified by CombiFlash chromatography to afford the title compound. ¹H NMR (400 MHz, DMSO-d₆): δ 7.31 (m, 1H), 6.94 (d, J=2.8 Hz, 1H), 6.87 (m, 1H), 6.78 (d, J=8.4 Hz, 1H), 6.71 (dd, J=8.4, 2.8 Hz, 1H), 6.50 (m, 1H), 5.09 (br s, 2H), 1.06 (s, 12H).

### Example S-4: Synthesis of 7-(2-(2,4-difluorophenoxy)-5-(ethylsulfonamido)phenyl)-N-ethyl-5-methyl-4-oxo-4,5-dihydrofuro[3,2-c]pyridine-2-carboxamide, Compound 65

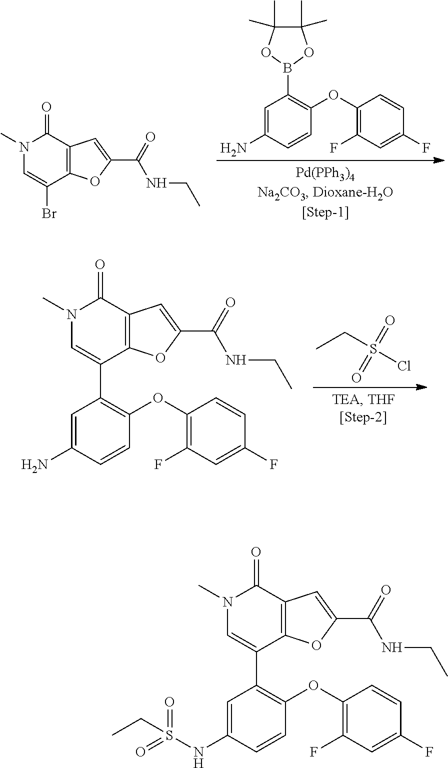

Step 1. Synthesis of 7-(5-amino-2-(2,4-difluorophenoxy)phenyl)-N-ethyl-5-methyl-4-oxo-4,5-dihydrofuro[3,2-c]pyridine-2-carboxamide: To a stirred solution of 7-bromo-N-ethyl-5-methyl-4-oxo-4,5-dihydrofuro[3,2-c]pyridine-2-carboxamide (0.15 g, 0.50 mmol, 1 eq) in 1,4-dioxane (4 mL) were added 4-(2,4-difluorophenoxy)-3-(4,4,5,5-tetramethyl-1,3,2-dioxaborolan-2-yl)aniline (0.23 g, 0.65 mmol, 1.3 eq) and Na₂CO₃ (0.13 g, 1.25 mmol, 2.5 eq) dissolved in water (1 mL) and the mixture was degassed under nitrogen for 20 min. Pd(PPh₃)₄ (0.018 g, 0.015 mmol, 0.03 eq) was then added to the mixture and the mixture was further degassed under nitrogen for 15 min. The resultant mixture was then heated at 100° C. for 4 h. The reaction was complete after 4 h. The mixture was cooled to RT and ice-cold water (30 mL) was added to obtain a precipitate which was filtered over Buchner funnel to obtain a crude residue which was purified by CombiFlash Chromatography to afford the title compound. LCMS: 440 [M+H]⁺.

Step 4. Synthesis of 7-(2-(2,4-difluorophenoxy)-5-(ethylsulfonamido)phenyl)-N-ethyl-5-methyl-4-oxo-4,5-dihydrofuro[3,2-c]pyridine-2-carboxamide: To a stirred solution of 7-(5-amino-2-(2,4-difluorophenoxy)phenyl)-N-ethyl-5-methyl-4-oxo-4,5-dihydrofuro[3,2-c] pyridine-2-carboxamide (0.1 g, 0.23 mmol, 1 eq) in THF (5 mL) was added triethylamine (0.07 g, 0.68 mmol, 3 eq) followed by ethanesulfonyl chloride (0.1 g, 0.8 mmol, 3.5 eq) at 0° C. and the resultant mixture was stirred at RT for 4 h. To the mixture was added water (20 mL) and it was with extracted with EtOAc (30 mL×2). The combined organic layers were washed with saturated NaHCO₃ solution (30 mL), brine (30 mL), dried over anhydrous Na₂SO₄, filtered and concentrated under reduced pressure to obtain a crude which was purified by reversed-phase HPLC to afford the title compound. LCMS: 532 [M+H]⁺; ¹H NMR (400 MHz, CD₃OD): δ 7.95 (s, 1H), 7.62 (d, J=2.6 Hz, 1H), 7.58 (s, 1H), 7.25 (dd, J=2.6, 8.8 Hz, 1H), 7.17 (d, J=5.7 Hz, 1H), 7.07 (br. s., 1H), 6.93 (d, J=8.8 Hz, 2H), 3.68 (s, 3H), 3.40 (q, J=7.2 Hz, 2H), 3.16 (q, J=7.2 Hz, 2H), 1.36 (t, J=7.5 Hz, 3H), 1.21 (t, J=7.2 Hz, 3H).

It is understood that compounds from the Table 1 are synthesized using the General Synthetic Schemes 1-4 or using the experimental procedures as described in the example S-1 to S-4 and the steps involved in the synthetic routes are clearly familiar to those skilled in the art, wherein the substituents described in compounds of the Formula (I), (II), (IIa-1) to (IIa-14), (III), (IIIa-1) to (IIIa-14), herein can be varied with a choice of appropriate starting materials and reagents utilized in the steps presented.

### BIOLOGICAL EXAMPLES

### Example B-1. Bromodomain and Extraterminal Domain (BET) Binding Assay

The bromodomain binding assays are performed by Reaction Biology Corp., Malvern, Pa., USA (www.reactionbiology.com). The BET binding assays are conducted in 384 well microplates in assay buffer (50 mM HEPES-HCl, pH 7.5, 100 mM NaCl, 1 mg/ml BSA, 0.05% CHAPS, and 0.5% DMSO) with compounds added as DMSO stocks at a single concentration or with 10-point dose response titrations. BET protein or assay buffer are delivered to the appropriate wells of the microplate. Test compound is then delivered by acoustic technology via a Labcyte Echo550 liquid handler. The microplate is centrifuged for 5 min and pre-incubated for 30 min at RT with gentle shaking. The ligand (histone H4 peptide (1-21) K5/8/12/16Ac-biotin) is delivered and the microplate is again centrifuged for 5 min and allowed to incubate for 30 min at RT with gentle shaking. Donor beads are then added in the absence of light and the microplate is centrifuged and gently shaken. After 5 min, acceptor beads are added in the absence of light and the microplate is centrifuged and gently shaken in the dark for 60 min. The microplate is read using a Perkin Elmer EnSpire Alpha plate reader (λ Ex/λ Em=680/520-620 nm). Percent inhibition is calculated relative to positive and negative controls on a per plate basis. For titration experiments, IC₅₀ values are determined by fitting the percent inhibition versus compound concentration.

Final Protein and Ligand Concentration

| Target | Protein Conc. (nM) | Ligand Conc. (nM) |
|---|---|---|
| BRD2-1 | 40 | 40 |
| BRD2-2 | 120 | 60 |
| BRD3-1 | 30 | 40 |
| BRD3-2 | 75 | 75 |
| BRD4-1 | 20 | 20 |
| BRD4-2 | 130 | 70 |
| BRD-T | 60 | 40 |

Compounds described herein were assayed and found to bind to bromodomain and extraterminal domain proteins BRD4-1 and BRD4-2. IC₅₀ for compounds of the invention are shown in Table 2.

TABLE 2
BRD4-1 and BRD4-2 IC₅₀ (μM)

| Synthesis | Compound | | |
|---|---|---|---|
| Example No. | No. | BRD4-1 | BRD4-2 |
| S-2 | 1 | 2.37 | 0.0007 |
| S-4 | 65 | 0.004 | 0.003 |

### Example B-2. Cell Viability Assays

The effects of test compounds are studied in a cell viability assay in the MV-4-11 human acute myeloid leukemia cell line. The cells are harvested during the logarithmic growth period and counted. Cells are seeded at a count of 15000 cells per well/100 l. After seeding, cells are incubated at 37° C., 5% CO₂ for 1 hr. Cells are treated with test compounds at 8 concentrations within a desired concentration range (e.g. 5 nM-10 PM) for generation of dose response curves by preparing serial dilutions of the test compound in DMSO which are further diluted with culture medium and then added to each well. The plate is further incubated for another 72 h in a humidified incubator at 37° C. and 5% CO₂. The assay is terminated by addition of Cell Titer-Glo reagent (Promega, Madison, Wis.) at ¼ the volume of total medium per well. Contents are mixed, the plate is incubated for 10 min at room temperature and luminescence is measured. Cell viability data are plotted using GraphPad Prism (GraphPad Software, Inc., San Diego, Calif.). In addition, a nonlinear regression model with a sigmoidal dose response and variable slope within GraphPad Prism is used to calculate the IC₅₀ value of individual test compounds.

The effects of test compounds are also studied in the IEC-6 rat intestinal epithelial cell line to assess potential toxicity to non-cancerous cells. The cells are harvested during the logarithmic growth period and counted. In Protocol A, cells are seeded at a count of 3000 cells per well/100 μl in a 96-well plate. After seeding, cells are incubated at 37° C., 5% CO₂ for 24 hr. Cells are treated with test compounds at 8 concentrations within a desired concentration range (e.g. 5 nM-10 μM) for generation of dose response curves by preparing serial dilutions of the test compound in DMSO which are further diluted with culture medium and then added to each well. The plate is further incubated for another 96 h in humidified incubator at 37° C. and 5% CO₂. The assay is terminated by addition of resazurin (#R7017, Sigma). The plate is incubated for 4 h at 37° C., 5% CO₂ and fluorescence is measured using excitation and emission wavelengths of 535 and 590 nm, respectively. Cell viability data are plotted using GraphPad Prism (GraphPad Software, Inc., San Diego, Calif.). In addition, a nonlinear regression model with a sigmoidal dose response and variable slope within GraphPad Prism is used to calculate the IC₅₀ value of individual test compounds. Protocol B is the same as Protocol A except that cells are seeded at a count of 4000 cells per well/100 μL in a 96-well plate, and the incubation with test compound is for 48 h instead of 96 h.

Other compounds of the disclosure are also assayed for effect on cell viability. In addition, a panel of BET-sensitive and insensitive cell lines is profiled for effect on cell viability using test compounds. Cells are cultured in the presence of inhibitors at various concentrations for up to 72 hr. For cell viability assays as previously described (Guo Y, et al. 2012. J Hematol Oncol 5:72; Chen Y, et al. 2016. Oncogene 35:2971-8), 0.08 mg/ml XTT (2,3-bis-(2-methoxy-4-nitro-5-sulfophenyl)-2H-tetrazolium-5-carboxanilide) and 8 μM phenazine methyl sulfate (PMS) are added to the cells at the end of the test compound or vehicle treatment duration, and absorbance at 450 nm is measured after 3 h incubation at 37° C. Assays are performed in triplicates. IC₅₀ values are estimated using a non-linear mixed effect model fitting a sigmoid curve to the experimental dose response data (Vis D J, et al. 2016. Pharmacogenomics 17(7):691-700).

### Example B-3 Histologic Analysis

The inhibitory effects of test compounds on the growth of cells are demonstrated by Wright-Giemsa staining of cells fixed to glass slides after incubation of the test compound or vehicle with the cells for a certain duration (e.g., 48 h). Morphologic changes of treated cells associated with cell cycle arrest, such as condensed nuclei and shrinking or swollen cell membranes are noted.

### Example B-4. In Vivo Efficacy Study

A study to evaluate test compound pharmacodynamics in MV-4-11 systemic leukemia model in NOD SCID mice is conducted. Female NOD SCID mice are inoculated with MV-4-11 cells systemically. Four weeks after cell inoculation, each animal is administered a single IV dose of test compound or vehicle. The dosing volume is 10 mL/kg (0.200 mL/20 g mouse), with volume adjusted according to body weight. Four hours after dosing, animals are sacrificed. Bone marrow and spleen (weight and size are recorded) are dissected, crushed in PBS and made into single cell suspensions for analysis by flow cytometry for the assessment of leukemic engraftment. Western blot analyses of bone marrow and spleen cell extracts with antibody against the housekeeping protein c-Myc are carried out for animals with successful leukemic engraftment.

### Example B-5. Mouse Xenograft Model

To examine the in vivo antitumor activity of test compound (as a single agent and in combination with other agents such as enzalutamide) in a castration resistant prostate cancer mouse model, tumor growth experiments are performed in a VCaP cell line mouse xenograft model. Cells are implanted subcutaneously into the flanks of 4-week old male immunodeficient mice (such as nude or SCID mice) and allowed to grow. Tumors are measured using a caliper and tumor volumes calculated using the formula: Tumor volume=(a×b²/2) where ‘b’ is the smallest diameter and ‘a’ is the largest diameter. Once the established tumors reach approximately 200 mm³, the tumor-bearing mice are surgically castrated. The mice are stratified into treatment groups once the tumors grow back to the pre-castration size. The treatment groups are, for example: vehicle control, enzalutamide alone, test compound alone, and enzalutamide+test compound at 10 mice per group. The exact treatment groups, drug dose, and dosing schedule are determined according to the specific needs of the study. Tumor growth is monitored, and volume recorded at regular intervals. When the individual tumor of each mouse reaches an approximate end-point (tumor volume >1,500 mm³), the mouse is sacrificed. The tumor growth inhibition (TGI) is calculated by comparing the control group's tumor measurements with the other study groups once the predetermined endpoint is reached in the control group.

Although the foregoing disclosure has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the disclosure.

1. A compound of Formula (I): 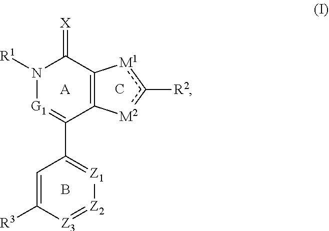
or a tautomer or isomer thereof, or a pharmaceutically acceptable salt of any of the foregoing, wherein:
each [symbol] is independently a single bond or double bond;
X is O or S;
R¹ is hydrogen, C₁-C₃ alkyl, —(C₁-C₃ alkylene)OH, C₁-C₃ haloalkyl, or C₃-C₄ cycloalkyl;
G₁ is CR^{a} or N, wherein:
R^{a} is hydrogen, halogen, C₁-C₆ alkyl, or C₁-C₆ haloalkyl;
Z₁ is C—W₁—R^{c}, wherein:
each W₁ is independently —O— or —NR^{w1}—, wherein:
R^{w1} is hydrogen, C₃-C₆ cycloalkyl, or C₁-C₄ alkyl optionally substituted by oxo, —OH, or halogen, and
R^{c} is independently C₃_C₆ cycloalkyl, 4- to 6-membered heterocyclyl, C₆-C₁₄ aryl, or 5- or 6-membered heteroaryl, each of which is independently optionally substituted by R^{c}, wherein each R^{c1} is independently halogen, C₁-C₄ alkyl, C₃-C₆ cycloalkyl, 3- to 6-membered heterocyclyl, cyano, oxo, C₁-C₄ alkoxy, C₁-C₄ haloalkoxy, C₁-C₄ haloalkyl, —OR¹⁰, —NR¹⁰R¹¹, —C(O)NR¹⁰R¹¹, —NR¹⁰C(O)R¹¹, —S(O)₂R¹⁰, —NR¹⁰S(O)₂R¹¹, or —S(O)₂NR¹⁰R¹¹;
Z₂ is C—W₂—R^{d} or N, wherein:
W₂ is —O—, —NR^{w2}—, or a bond, wherein:
R^{w2} is hydrogen, C₃-C₆ cycloalkyl, or C₁-C₄ alkyl optionally substituted by oxo, —OH, or halogen, and
R^{d} is independently hydrogen, halogen, cyano, 3- to 6-membered heterocyclyl, or C₁-C₄ alkyl;
Z₃ is C—R^{e} or N, wherein:
R^{e} is independently hydrogen, halogen, cyano, 3- to 6-membered heterocyclyl, or C₁-C₄ alkyl;
M¹ is O or CR^{1a};
M² is O or CR^{2a}, provided that
(1) when M¹ is O, then the [symbol] adjacent to M¹ is a single bond and the [symbol] adjacent to M² is a double bond,
(2) when M² is O, then the [symbol] adjacent to M² is a single bond and the [symbol] adjacent to M¹ is a double bond, and
(3) at least one of M¹ and M² is O;
R^{1a} and R^{2a} are each independently hydrogen, halogen, C₁-C₄ alkyl, C₃-C₆ cycloalkyl, 3- to 6-membered heterocyclyl, 5- to 10-membered heteroaryl, cyano, C₁-C₄ haloalkyl, C₁-C₄ alkoxy, C₁-C₄ haloalkoxy, —OR¹⁰, —NR¹⁰R¹¹, —C(O)OR¹⁰, —C(O)NR¹⁰R¹¹, —NR¹⁰C(O)R¹¹, —S(O)₂R¹⁰, —NR¹⁰S(O)₂R¹¹ or —S(O)₂NR¹⁰R¹¹, each of which is independently optionally substituted by R¹²;
R² is hydrogen, halogen, C₃-C₆ cycloalkyl, 3- to 6-membered heterocyclyl, 5- to 10-membered heteroaryl, cyano, C₁-C₄ alkoxy, C₁-C₄ haloalkoxy, —OR¹⁰, —NR¹⁰R¹¹, —C(O)OR¹⁰, —C(O)NR¹⁰R¹¹, —NR¹⁰C(O)R¹¹, —S(O)₂R¹⁰, —NR¹⁰S(O)₂R¹¹, or —S(O)₂NR¹⁰R¹¹, each of which is independently optionally substituted by R¹²;
R³ is —(CH₂)ₘNR¹³S(O)₂R¹⁴, wherein m is 0, 1, 2 or 3; C₃-C₆ cycloalkyl optionally substituted by halogen, oxo, —CN, or —OH; or C₁-C₄ alkyl substituted by halogen, oxo, —CN, or —OH, provided that when R³ is —(CH₂)ₘNR¹³S(O)₂R¹⁴, then R² is halogen, C₃-C₆ cycloalkyl, 3- to 6-membered heterocyclyl, 5- to 10-membered heteroaryl, cyano, C₁-C₄ alkoxy, C₁-C₄ haloalkoxy, —OR¹⁰, —NR¹⁰R¹¹, —C(O)OR¹⁰, —C(O)NR¹⁰R¹¹, —NR¹⁰C(O)R¹¹, —S(O)₂R¹⁰, —NR¹⁰S(O)₂R¹¹, or —S(O)₂NR¹⁰R¹¹, each of which is independently optionally substituted by R¹²;
R¹⁰ and R¹¹ are each independently hydrogen, C₁-C₄ alkyl, C₁-C₄ alkoxy, C₁-C₄ alkenyl, C₃-C₆ cycloalkyl, 3- to 6-membered heterocyclyl, —(C₁-C₃ alkylene) C₃-C₆ cycloalkyl, —(C₁-C₃ alkylene) 3- to 6-membered heterocyclyl, —NR¹⁵R¹⁶, or —C(O)R¹², wherein each of R¹⁰ and R¹¹ is independently optionally substituted by halogen, oxo, —CN, —CF₃, —OH, —NR¹³R¹⁴, —C(O)NR¹³R¹⁴, or C₁-C₄ alkyl optionally substituted by halogen, oxo, —CN, —CF₃, or —OH,
or R¹⁰ and R¹¹ are taken together with the atom or atoms to which they are attached to form a 3- to 6-membered heterocyclyl ring optionally substituted by halogen, oxo, —CN, —CF₃, —OH, or C₁-C₄ alkyl optionally substituted by halogen, oxo, —CN, or —OH;
each R¹² is independently halogen, cyano, C₁-C₄ haloalkyl, C₁-C₄ alkoxy, C₁-C₄ haloalkoxy, —OR¹⁵, —NR¹⁵R¹⁶, —C(O)NR¹⁵R¹⁶, —NR¹⁵(O)R¹⁶, —S(O)₂R¹⁵, —NR¹⁵(O)₂R¹⁶, —S(O)₂NR¹⁵R¹⁶, C₃-C₆ cycloalkyl, 3- to 6-membered heterocyclyl, or C₁-C₄ alkyl, each of which is independently optionally substituted by halogen, oxo, —CF₃, —CN, —OH, —NR¹³R¹⁴, or —NR¹³C(O)R¹⁴;
R¹³ and R¹⁴ are independently hydrogen, C₁-C₄ alkyl C₃-C₆ cycloalkyl, or 3- to 6-membered heterocyclyl, each of which is independently optionally substituted by halogen, oxo, —CN, or —OH,
or R¹³ and R¹⁴ are taken together with the atom or atoms to which they are attached to form a 3- to 6-membered heterocyclyl ring optionally substituted by halogen, oxo, —CN, —OH, or C₁-C₄ alkyl optionally substituted by halogen, oxo, —CN, or —OH; and
each R¹⁵ and R¹⁶ are independently hydrogen, C₁-C₄ alkyl, C₃-C₆ cycloalkyl, or 3- to 6-membered heterocyclyl, each of which is independently optionally substituted by halogen, oxo, —CN, or —OH,
or R¹⁵ and R¹⁶ are taken together with the atoms to which they are attached to form a 3- to 6-membered heterocyclyl ring optionally substituted by halogen, oxo, —CN, —OH, or C₁-C₄ alkyl optionally substituted by halogen, oxo, —CN, or —OH.

2. The compound of claim 1, or a tautomer or isomer thereof, or a pharmaceutically acceptable salt of any of the foregoing, wherein M¹ is O.

3. The compound of claim 1, or a tautomer or isomer thereof, or a pharmaceutically acceptable salt of any of the foregoing, wherein M² is O.

4. The compound of any one of claims 1-3, or a tautomer or isomer thereof, or a pharmaceutically acceptable salt of any of the foregoing, wherein R² is —C(O)NR¹⁰R¹¹ or 5- to 10-membered heteroaryl, wherein the —C(O)NR¹⁰R¹¹ and 5- to 10-membered heteroaryl of R² are each optionally substituted by R¹².

5. The compound of any one of claims 1-4, or a tautomer or isomer thereof, or a pharmaceutically acceptable salt of any of the foregoing, wherein R² is —C(O)NR¹⁰R¹¹.

6. The compound of claim 4 or 5, or a tautomer or isomer thereof, or a pharmaceutically acceptable salt of any of the foregoing, wherein R¹⁰ and R¹¹ are each independently hydrogen or C₁-C₄ alkyl.

7. The compound of claim 6, or a tautomer or isomer thereof, or a pharmaceutically acceptable salt of any of the foregoing, wherein R² is

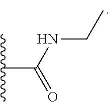 [SMILES: *C(=O)NCC]

8. The compound of any one of claims 1-4, or a tautomer or isomer thereof, or a pharmaceutically acceptable salt of any of the foregoing, wherein R² is 5- to 10-membered heteroaryl optionally substituted by R¹².

9. The compound of claim 8, or a tautomer or isomer thereof, or a pharmaceutically acceptable salt of any of the foregoing, wherein R² is 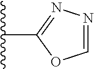 [SMILES: *c1nnco1]
which is optionally substituted by R¹².

10. The compound of claim 9, or a tautomer or isomer thereof, or a pharmaceutically acceptable salt of any of the foregoing, wherein R¹² is C₁-C₄ alkyl.

11. The compound of claim 10, or a tautomer or isomer thereof, or a pharmaceutically acceptable salt of any of the foregoing, wherein R² is

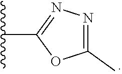 [SMILES: *c1nnc(C)o1]

12. The compound of any one of claims 1-11, or a tautomer or isomer thereof, or a pharmaceutically acceptable salt of any of the foregoing, wherein X is O.

13. The compound of any one of claims 1-12, or a tautomer or isomer thereof, or a pharmaceutically acceptable salt of any of the foregoing, wherein Z₁ is C—O—R^{c}.

14. The compound of claim 13, or a tautomer or isomer thereof, or a pharmaceutically acceptable salt of any of the foregoing, wherein R^{c} is phenyl, pyridinyl, or cyclohexyl, each of which is independently optionally substituted by R^{c1}.

15. The compound of claim 13 or 14, or a tautomer or isomer thereof, or a pharmaceutically acceptable salt of any of the foregoing, wherein R^{c} is 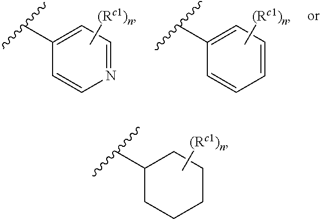
wherein n is 0, 1, 2, 3, or 4.

16. The compound of claim 14 or 15, or a tautomer or isomer thereof, or a pharmaceutically acceptable salt of any of the foregoing, wherein each R^{c1} is independently halogen or C₁-C₄ alkyl.

17. The compound of claim 16, or a tautomer or isomer thereof, or a pharmaceutically acceptable salt of any of the foregoing, wherein R^{c} is

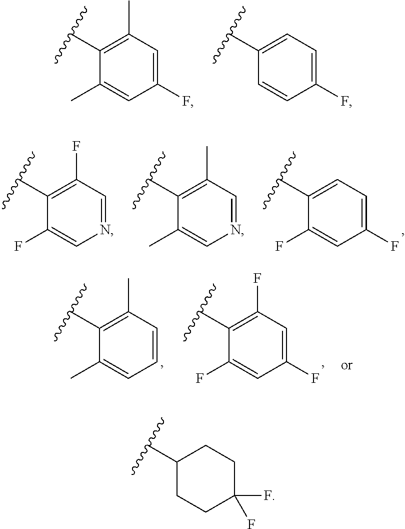

18. The compound of any one of claims 1-17, or a tautomer or isomer thereof, or a pharmaceutically acceptable salt of any of the foregoing, wherein Z₂ is CH.

19. The compound of any one of claims 1-17, or a tautomer or isomer thereof, or a pharmaceutically acceptable salt of any of the foregoing, wherein Z₂ is N.

20. The compound of any one of claims 1-19, or a tautomer or isomer thereof, or a pharmaceutically acceptable salt of any of the foregoing, wherein Z₃ is CH.

21. The compound of any one of claims 1-20, or a tautomer or isomer thereof, or a pharmaceutically acceptable salt of any of the foregoing, wherein R¹ is C₁-C₃ alkyl.

22. The compound of claim 21, or a tautomer or isomer thereof, or a pharmaceutically acceptable salt of any of the foregoing, wherein R¹ is methyl.

23. The compound of any one of claims 1-22, or a tautomer or isomer thereof, or a pharmaceutically acceptable salt of any of the foregoing, wherein G₁ is CR^{a}, wherein R^{a} is hydrogen.

24. The compound of claim 1, or a tautomer or isomer thereof, or a pharmaceutically acceptable salt of any of the foregoing, wherein the compound is selected from the compounds in Table 1.

25. A pharmaceutical composition comprising the compound of any one of claims 1-24, or a tautomer or isomer thereof, or a pharmaceutically acceptable salt of any of the foregoing, and a pharmaceutically acceptable carrier.

26. A method of treating disease mediated by bromodomain and extraterminal domain (BET) in an individual in need thereof comprising administering to the individual a therapeutically effective amount of the compound of any one of claims 1-24, or a tautomer or isomer thereof, or a pharmaceutically acceptable salt of any of the foregoing.

27. A method of treating cancer in an individual in need thereof comprising administering to the individual a therapeutically effective amount of the compound of any one of claims 1-24, or a tautomer or isomer thereof, or a pharmaceutically acceptable salt of any of the foregoing.

28. A method of inhibiting bromodomain and extraterminal domain (BET) in a cell, comprising administering the compound of any one of claims 1-24, or a tautomer or isomer thereof, or a pharmaceutically acceptable salt of any of the foregoing, to the cells.

29. Use of the compound of any one of claims 1-24, or a tautomer or isomer thereof, or a pharmaceutically acceptable salt of any of the foregoing, in the manufacture of a medicament for treatment of a disease mediated by bromodomain and extraterminal domain (BET).

30. A kit comprising the compound of any one of claims 1-24, or a tautomer or isomer thereof, or a pharmaceutically acceptable salt of any of the foregoing.
